(19) United States
(12) Patent Application Publication
(10) Pub. No.: US 2007/0020660 A1
(43) Pub. Date: Jan. 25, 2007

(54) Expression profiles of peripheral blood mononuclear cells for inflammatory bowel diseases

(76) Inventors: Michael E. Burczynski, Collegeville, PA (US); Ron L. Peterson, North Chelmsford, MA (US); Natalie C. Twine, Goffstown, NH (US); Andrew Strahs, Maynard, MA (US); Frederick W. Immermann, Suffern, NY (US); Ullrich Schwertschlag, Beverly Farms, MA (US); Monette M. Cotreau, Acton, MA (US); Andrew J. Dorner, Lexington, MA (US)

Correspondence Address: FITZPATRICK CELLA (WYETH), 30 ROCKEFELLER PLAZA, NEW YORK, NY, 10112-3800, US

(21) Appl. No.: 11/448,611

(22) Filed: Jun. 6, 2006

Related U.S. Application Data
(60) Provisional application No. 60/687,331, filed on Jun. 6, 2005.
(60) Provisional application No. 60/692,295, filed on Jun. 20, 2005.

(51) Int. Cl. C12Q 1/68; C12P 19/34

(52) U.S. Cl. 435/6; 435/91.2

(57) ABSTRACT

The present invention is directed to the identification of PBMC- and IBD-associated biomarkers that may be used to diagnose inflammatory bowel disease, and optionally, distinguish between PBMCs isolated from a patient with Crohn's disease and PBMCs isolated from a patient with ulcerative colitis. The present invention is further directed to methods of screening, including high throughput methods of screening, for regulatory agents capable of regulating the activity of PBMC- and IBD-associated biomarkers. Provided are compositions of PBMC- and IBD-associated biomarkers, including regulatory agents of at least one PBMC- and IBD-associated biomarker for methods of diagnosis, prognosis, therapeutic intervention and prevention of an inflammatory bowel disease, e.g., Crohn's disease and ulcerative colitis. Moreover, the present invention is also directed to methods that can be used to assess the efficacy of test compounds and therapies in the treatment inflammatory bowel disease, i.e., Crohn's disease or ulcerative colitis.

This application claims the benefit of priority from U.S. Provisional Patent Application No. 60/687,331, filed Jun. 6, 2005, and U.S. Provisional Patent Application No. 60/692,295, filed Jun. 20, 2005; the contents of both applications are hereby incorporated by reference herein in their entireties.

### BACKGROUND OF THE INVENTION

1. Field of the Invention

The invention is directed to the analysis of expression profiles of peripheral blood mononuclear cells (PBMCs) isolated from patients with inflammatory bowel disease and the identification of PBMC transcriptional gene signatures capable of distinguishing between patients suffering from one of two types of inflammatory bowel disease, i.e., Crohn's disease and ulcerative colitis.

2. Related Background Art

Ulcerative colitis (UC) and Crohn's disease (CD) are two common, chronic, and relapsing inflammatory bowel diseases (IBDs) that share several demographic and clinical characteristics. However, UC and CD present key differences in tissue damage, which suggests distinct etiopathogenic processes for the two diseases. One proposed etiology of IBD in general is the inappropriate activation of the mucosal immune system against normal intestinal luminal bacterial flora (Podolsky (2002) N. Engl. J Med. 347:417-29). A transmural, granulomatous inflammatory process associated with Thl-type responses is characteristic of CD, whereas inflammation in UC tends to be limited to the mucosa and contains large numbers of immunoglobulin-secreting plasma cells that appear to be associated with Th2 responses (Podolsky, supra). Both diseases are complex disorders in which a combination of environmental and genetic factors may determine the susceptibility of an individual to disease (Bouma and Strober (2003) Nat. Rev. Immunol. 3:521-33).

The ability to quantitate the global expression profiles at the level of RNA using oligonucleotide microarrays has recently been applied to investigate transcriptional signatures present in surgically resected gastrointestinal tissue obtained from CD and UC patients (Lawrance et al. (2001) Hum. Mol. Genet. 10:445-56; see also Warner and Dieckgraefe (2002) Inflamm. Bowel. Dis. 8:140-57). These studies identified genes involved in inflammatory responses generally altered in IBD. Additionally, the studies showed that the gastrointestinal tissue transcriptomes obtained from UC and CD patients are quite distinct, with gene sets identified that appear to distinguish UC tissue from CD tissue.

In contrast to gastrointestinal tissue from surgical resections or biopsies, peripheral blood is a much more accessible tissue source of cells that might be used to distinguish between UC and CD. Circulating peripheral blood mononuclear cells (PBMCs) are responsible for the comprehensive surveillance of the body for signs of infection and disease. PBMCs may therefore serve as a surrogate tissue for evaluation of disease-induced gene expression as a marker of disease status or severity (for a general review see Rockett et al. (2004) Toxicol. Appl. Pharmacol. 194:189-99). Maas and coworkers identified PBMC profiles common to patients with autoimmune diseases such as rheumatoid arthritis, systemic lupus erythematosus, type I diabetes, and multiple sclerosis (Maas et al. (2002) J Immunol. 169:5-9). Twine and coworkers have shown that, in the context of a nonautoimmune disease, PBMCs obtained from renal cell carcinoma (RCC) patients exhibit disease-associated transcriptomes distinct from those of healthy volunteers (Twine et al. (2003) Cancer Res. 63:6069-75). Mannick and coworkers recently explored expression profiles of PBMCs from seven CD patients and five UC patients with a 2400 gene cDNA microarray and described several genes that appear differentially expressed between these diseases (Mannick et al. (2004) Clin. Immunol. 112:247-57); previously, other genes were reported as regulated in peripheral blood mononuclear cells of Crohn's patients at the mRNA level (Gijsbers et al. (2004) Eur. J Immunol. 34:1992-2000; Hori et al. (2002) J Gastroenterol. Hepatol. 17:1070-77; Gonsky et al. (1998) J Immunol. 160: 4914-22).

Although peripheral inflammatory components are proposed to be involved in both forms of IBD, the transcriptional gene profiles of circulating PBMCs from healthy patients and patients with histologically verified diagnoses of IBD, either in the form of CD or UC, have not yet been successfully used to develop gene classifiers that allow distinction between disorders. To date, the ability of PBMC-associated transcriptomes to diagnose IBD and/or differentiate between CD and UC has been unknown in the art.

The present invention solves this problem by determining whether gene expression patterns in PBMCs of patients with CD and UC are sufficiently distinct to enable their classification on the basis of gene expression profiles in PBMCs alone, and by providing PBMC- and IBD-associated transcriptional gene expression patterns that may be used to distinguish patients with IBD from healthy subjects, and optionally, patients with CD from patients with UC. Thus, the diagnosis, prognosis, and/or monitoring of inflammatory bowel disease, and/or of different forms of IBD, i.e., CD and UC, may be assisted by the relatively noninvasive methods of the invention involving the transcriptional profiling of peripheral blood mononuclear cells from patients.

### SUMMARY OF THE INVENTION

In one aspect, the present invention is based on the identification and categorization of a number of PBMC- and IBD-associated biomarkers (e.g., the PBMC- and IBD-associated biomarkers listed in Tables 1-4, which are differentially expressed in 1) PBMCs of patients with inflammatory bowel disease compared to PBMCs of subjects substantially free of IBD, e.g., healthy subjects, 2) PBMCs of patients with Crohn's disease compared to PBMCs of subjects substantially free of IBD, e.g., healthy subjects, 3) PBMCs of patients with ulcerative colitis compared to PBMCs of subjects substantially free of IBD, e.g., healthy subjects, and 4) PBMCs of patients with Crohn's disease compared to patients with ulcerative colitis, respectively). The PBMC- and IBD-associated biomarkers provided by the invention and listed in Tables 1-4 are also categorized into Group I, Group II, Group III, and Group IV, respectively, based on whether they may be optimally used to diagnose, prognose, or monitor the progress of 1) a patient with IBD in the form of either Crohn's disease or ulcerative colitis (Group I biomarkers; also referred to herein as a set of “common biomarkers”); 2) a patient with Crohn's disease (Group II biomarkers; also referred to herein as a set of “CD biomarkers”); or 3) a patient with ulcerative colitis (Group III biomarkers; also referred to herein as a set of “UC biomarkers”); and/or optimally used to differentiate whether a patient with IBD has Crohn's disease or ulcerative colitis (Group IV; also referred to herein as a set of“CDvUC biomarkers”). In addition, the PBMC- and IBD-associated biomarkers listed in Table 5 and categorized as Group V biomarkers (also referred to herein as a set of “classifying biomarkers”) may also be used to distinguish a patient with Crohn's disease from a patient with ulcerative colitis. These PBMC- and IBD-associated biomarkers may, in turn, also be components of IBD disease pathways, and thus, may serve as novel therapeutic targets for treatment of inflammatory bowel disease, i.e., Crohn's disease or ulcerative colitis.

Accordingly, the present invention pertains to polynucleotides, the polypeptides they encode, and fragments, homologs and isoforms thereof, as PBMC- and IBD-associated biomarkers (which may be categorized as Group I, Group II, Group III, Group IV, and/or Group V biomarkers) for inflammatory bowel disease, Crohn's disease, and/or ulcerative colitis. The invention also pertains to the use of antibodies directed against the PBMC- and IBD biomarkers of the invention, arrays comprising the biomarkers of the invention, and/or assays involving the biomarkers of the invention (e.g., microarray assays, Q-PCR assays, nucleic reporter assays, etc.). Additionally, the present invention pertains to the use of expression profiles of these PBMC- and IBD-associated biomarkers to indicate the presence of, or a risk for, inflammatory bowel disease, Crohn's disease and/or ulcerative colitis. With respect to an inflammatory bowel disease, Crohn's disease, and/or ulcerative colitis, these PBMC- and IBD-associated biomarkers are also useful to correlate differences in levels of expression with a poor or favorable prognosis. The PBMC- and IBD-associated biomarkers may also be useful for assessing the efficacy of a treatment or therapy for an IBD. With respect to treatment for an IBD, e.g., Crohn's disease, ulcerative colitis, etc., the PBMC- and IBD-associated biomarkers of the invention may also be useful to screen for test compounds capable of ameliorating an IBD, and/or as therapeutic agents themselves.

In one aspect, the invention provides PBMC- and IBD-associated biomarkers whose level of expression, which signifies their quantity or activity, is correlated with the presence of inflammatory bowel disease, e.g., Crohn's disease or ulcerative colitis. The PBMC- and IBD-associated biomarkers of the invention may be polynucleotides (e.g., DNA, cDNA, mRNA), the polypeptides encoded by such polynucleotides, and fragments, homologs, and isoforms of such polynucleotides or polypeptides. In certain embodiments, the methods of the invention are performed by detecting the presence of a transcribed polynucleotide, or a portion thereof, wherein the transcribed polynucleotide comprises a PBMC- and IBD-associated biomarker. Alternatively, detection may be performed by detecting the presence of a protein, which corresponds to (i.e., is encoded by) the PBMC- and IBD-associated biomarker gene or RNA species. These methods may also be performed on the protein level; that is, protein expression levels of the PBMC- and IBD-associated biomarker proteins can be evaluated for diagnostic, prognostic and/or monitoring purposes, or to screen test compounds, or as therapeutic agents.

In some embodiments, panels comprising more than one PBMC- and IBD-associated biomarker(s) are used in the methods of the invention. In one embodiment, the invention provides a panel comprising a plurality of PBMC- and IBD-associated biomarkers. In one embodiment, a panel of the invention comprises at least two PBMC- and IBD-associated biomarkers. In one embodiment, a panel of the invention comprises at least three PBMC- and IBD-associated biomarkers. In one embodiment, a panel of the invention comprises at least four PBMC- and IBD-associated biomarkers. In one embodiment, a panel of the invention comprises at least five PBMC- and IBD-associated biomarkers. In one embodiment, a panel of the invention comprises at least six PBMC- and IBD-associated biomarkers. In one embodiment, a panel of the invention comprises at least seven PBMC- and IBD-associated biomarkers. In one embodiment, a panel of the invention comprises at least eight PBMC- and IBD-associated biomarkers. In one embodiment, a panel of the invention comprises at least nine PBMC- and IBD-associated biomarkers. In one embodiment, a panel of the invention comprises at least ten PBMC- and IBD-associated biomarkers. In one embodiment, a panel of the invention comprises at least eleven PBMC- and IBD-associated biomarkers. In one embodiment, a panel of the invention comprises at least twelve PBMC- and IBD-associated biomarkers. In other embodiments, the panel of biomarkers comprises common biomarkers, CD biomarkers, UC biomarkers, CDvUC biomarkers, and/or classifying biomarkers. Panels of biomarkers comprising biomarkers selected from Group I biomarkers, Group II biomarkers, Group III biomarkers, Group IV biomarkers, and/or Group V biomarkers are also provided. A skilled artisan will recognize that a panel of the invention may comprise any number and any combination of PBMC- and IBD-associated biomarkers of the invention, particularly the Group V biomarkers of the invention. Thus, in other nonlimiting embodiments of the invention, a panel of the invention comprises at least two, at least three, at least four, at least five, at least six, at least seven, at least eight, at least nine, at least ten, at least eleven, or at least twelve classifying biomarkers, e.g., the classifying biomarkers of Group V. For example, a nonlimiting panel of the invention may comprise the immunoglobulin heavy constant gamma 1 and immunoglobulin kappa constant biomarkers. Another nonlimiting panel of the invention may comprise the human 28S ribosomal RNA 5′region, protein tyrosine phosphatase receptor type C-associated protein, H3 histone family member K, integrin beta 3 (platelet glycoprotein IIIa, antigen CD61), and H2B histone family member Q biomarkers. Another nonlimiting panel of the invention may comprise the immunoglobulin heavy constant gamma 1, granzyme K, mutL homolog 3, lipocalin 2, CXCL5, serum deprivation response phosphatidylserine binding protein, and H3 histone family member K biomarkers. In one embodiment, a panel of the invention provides at least 70% accuracy (more preferably, at least 80% accuracy, most preferably at least 90% accuracy) (a) in determining whether a patient has (1) IBD in the form of either Crohn's disease or ulcerative colitis, (2) Crohn's disease, and/or (3) ulcerative colitis, and/or (b) in distinguishing whether a patient with IBD has Crohn's disease or ulcerative colitis. In another aspect of the invention, the expression levels of more than one PBMC- and IBD-associated biomarkers of the invention are determined in a particular subject sample for which information is desired (e.g., for diagnosis, prognosis, monitoring the course of treatment and/or disease, etc.).

In certain embodiments, a comparison of relative levels of expression of at least one PBMC- and IBD-associated biomarker is indicative of the severity of inflammatory bowel disease, Crohn's disease, and/or ulcerative colitis, and such a comparison permits for diagnostic, prognostic, and monitoring analysis. For example, comparison of expression of PBMC- and IBD-associated biomarker profiles of various disease progression states for IBD (and/or either UC or CD) provides a method for long-term prognosing, including the predicted duration of an outbreak or episode of either of these diseases. In another example, the evaluation of a particular treatment regimen may be evaluated, including whether a particular drug will act to improve the long-term prognosis in a particular patient.

A PBMC- and IBD-associated biomarker may also be useful as a target for a treatment or therapeutic agent. Therefore, without limitation as to mechanism, some of the methods of the invention are based, in part, on the principle that regulation of the expression of the PBMC- and IBD-biomarkers of the invention may ameliorate an inflammatory bowel disease when they are expressed at levels similar or substantially similar to those of PBMCs isolated from subjects substantially free of IBD, e.g., healthy subjects. The discovery of these differential expression patterns for individual PBMC- and IBD-associated biomarkers, or panels of such biomarkers, allows for screening of test compounds with the goal of regulating a particular expression pattern; for example, screening can be done for compounds that will convert an expression profile for a poor prognosis to one for a better or improved prognosis.

In relation to these embodiments, some PBMC- and IBD-associated biomarkers may comprise biomarkers that are determined to have modulated activity or expression in response to a therapy regimen. Alternatively, the modulation of the activity or expression of a PBMC- and IBD-associated biomarker may be correlated with the diagnosis or prognosis of inflammatory bowel disorder, Crohn's disease and/or ulcerative colitis. In addition, regulatory agents of the invention, e.g., regulatory agents of at least one PBMC- and IBD-associated biomarker (e.g., PBMC- and IBD-associated polynucleotides and/or polypeptides, related PBMC- and IBD-associated polynucleotides and/or polypeptides (e.g., inhibitory polynucleotides, inhibitory polypeptides (e.g., anti-biomarkers antibodies)), small molecules, etc.) may be administered as therapeutic drugs. In another embodiment of the invention, a regulatory agent of the invention may be used in combination with one or more other therapeutic compositions of the invention. Formulation of such compounds into pharmaceutical compositions is described below. Administration of such a therapeutic regulatory agent may regulate the aberrant expression of at least one PBMC- and IBD-associated biomarker, and therefore may be used to ameliorate or inhibit inflammatory bowel disease, Crohn's disease, and/or ulcerative colitis. In another embodiment of the invention, one or more regulatory agents or other therapeutic compositions of the invention may be used in combination with one or more other known therapeutic agents or compositions.

### BRIEF DESCRIPTION OF THE DRAWINGS

FIG. 1. Functional annotation and categories of transcripts identified as CD-associated, UC-associated, and differentially expressed between UC and CD. A) Shown are canonical pathways (x-axis) overrepresented in the CD versus normal ANCOVA comparison (gray bars), the UC versus normal ANCOVA comparison (black bars), and the UC versus CD ANCOVA comparison (white bars). In these panels, the negative log of the p-value (y-axis) is plotted in order to highlight more significant associations. The pathways interrogated (x-axis) are as follows: (1) amyloid processing, (2) apoptosis signaling, (3) arginine and proline metabolism, (4) B cell receptor signaling (nonimmunoglobulin), (5) cardiac β-adrenergic signaling, (6) chemokine signaling, (7) death receptor signaling, (8) ERK/MAPK signaling, (9) fatty acid metabolism, (10) cell cycle regulation (G1/S), (11) cell cycle regulation (G2/M), (12) G protein-coupled receptor signaling, (13) glutamate metabolism, (14) histidine metabolism, (15) IGF-1 signaling, (16) IL-2 signaling, (17) IL-4 signaling, (18) inositol phosphate metabolism, (19) insulin receptor signaling, (20) integrin signaling, (21) interferon signaling, (22) JAK/STAT signaling, (23) NF-κB signaling, (24) nitrogen metabolism, (25) p38 MAPK signaling, (26) PI3K/AKT signaling, (27) PPAR signaling, (28) prostaglandin and leukotriene metabolism, (29) purine metabolism, (30) pyrimidine metabolism, (31) starch and sucrose metabolism, (32) T cell receptor signaling, (33) tryptophan metabolism, (34) tyrosine metabolism, and (35) VEGF signaling. B) CD-specific transcripts in PBMCs were functionally annotated using the Ingenuity pathway analysis system (Ingenuity, Mountain View, Calif.); the relative distribution of transcripts in each of the chosen functional categories for the CD-associated genes are presented in the pie chart. C) UC-specific transcripts in PBMCs were functionally annotated manually and the relative distribution of transcripts in immunoglobulin versus nonimmunoglobulin categories is presented.

FIG. 2. Supervised class prediction of CD and UC using PBMC profiles. A) The relative overall accuracy (▪; y-axis), accuracy of CD classification (-•-; y-axis), and accuracy of UC classification (▴; y-axis) for panels consisting of 2-20 gene classifiers (x-axis) is shown. B) Shown are results of weighted voting class assignment in the test set of samples. Confidence scores in favor of CD are presented as positive values and confidence scores of the class assignments in favor of UC are presented as negative values. The overall accuracy of class assignment was 100% in the test set, where 14 of 14 Crohn's patients were correctly classified as Crohn's, 6 of 6 UC patients were correctly classified as UC, based solely upon expression patterns in PBMCs as obtained via microarray analysis. The actual origins of the PBMC profiles are indicated (CD patients=first fourteen white bars; UC patients=last six black bars).

FIG. 3. Real-time PCR confirmation of classifier transcript levels in CD and UC sample sets. A) Shown are average fold elevation (y-axis) of gene classifier transcripts (x-axis) detected as upregulated in CD as detected by Affymetrix microarray hybridization (Affymetrix; white columns) or quantitative real time RT-PCR (TAQMAN®; black columns). B) Shown are average fold elevation (y-axis) of gene classifier transcripts (x-axis) detected as upregulated in UC as detected by Affymetrix microarray hybridization (Affymetrix; white columns) or quantitative real-time RT-PCR (TAQMAN®; black columns).

FIG. 4. Comparison of discriminant and logistic analysis on classification of patients with either Crohn's disease or Ulcerative colitis using transcriptional profiles from Q-PCR analysis. Shown is the accuracy (y-axis) of logistic analysis or discriminant analysis for (A) twenty training sets or (B) twenty associated test sets.

### DETAILED DESCRIPTION OF THE INVENTION

Although expression profile analysis of gastrointestinal tissue biopsies have identified the presence of gastrointestinal-associated transcriptomes that may be used to distinguish two inflammatory bowel diseases, i.e., Crohn's disease (CD) or ulcerative colitis (UC), the required biopsy of gastrointestinal tissue makes such methods of diagnosis unattractive. Compared to gastrointestinal tissue biopsies, cells in the peripheral blood, in particular, circulating peripheral blood mononuclear cells (PBMCs), are much more accessible. Since PBMCs are responsible for the comprehensive surveillance of the body for signs of infection and disease, and IBDs apparently involve inflammatory processes, PBMCs may serve as surrogates to gastrointestinal tissue for evaluation of tissue- and disease-associated transcriptomes that may be useful for determining the status or severity of an IBD.

The present invention is directed to the utilization of at least one “transcriptional gene signature” (also referred to herein as a “gene signature,” “expression signature,” “transcriptome,” “profile,” or “gene profile”) of PBMCs, i.e., PBMC-associated transcriptional gene signatures, i.e., expression profiles of PBMCs, to determine whether a patient is suffering from an inflammatory bowel disease. The present invention is also directed to the use of PBMC-associated transcriptomes for the optional determination of whether a patient with IBD is suffering from Crohn's disease or ulcerative colitis. The present invention is based on the finding of PBMC-associated and IBD-associated, e.g., Crohn's disease-associated and/or ulcerative colitis-associated, transcriptomes. In particular, the invention is based on the identification of PBMC- and IBD-associated biomarkers, which may be categorized into five groups (Group I, Group II, Group III, Group IV and Group V) based on their utility in the diagnosis, prognosis, monitoring, and/or treatment of IBD, Crohn's disease and/or ulcerative colitis.

As used herein, the term “biomarker,” “gene classifier,” or “PBMC- and IBD-associated biomarker,” or the like, includes a polynucleotide (e.g., gene, transcript, EST, etc.) or polypeptide molecule that is substantially modulated (i.e., upregulated or downregulated) in quantity in peripheral blood mononuclear cells of subjects with inflammatory bowel disease (i.e., Crohn's disease and/or ulcerative colitis) as compared to a subject substantially free of IBD (e.g., a healthy subject). In certain embodiments, the PBMC- and IBD-associated biomarkers of the invention include the polynucleotides, their corresponding gene products, and fragments, homologs and isoforms thereof, of Group I biomarkers (also referred to as “common biomarkers”), Group II biomarkers (also referred to as “Crohn's disease-associated biomarkers,” “CD-associated biomarkers,” “CD-specific biomarkers,” or “CD biomarkers”), Group III biomarkers (also referred to as “ulcerative colitis-associated biomarkers,” “UC-associated biomarkers,” “UC-specific biomarkers” or “UC biomarkers”), Group IV biomarkers (also referred to as “CDvUC biomarkers”), and Group V biomarkers (also referred to as “classifying biomarkers”).

A PBMC- and IBD-associated biomarker of the invention may be a polynucleotide, its corresponding gene product, and fragments, homologs and isoforms thereof, that is substantially modulated (i.e., upregulated or downregulated) in PBMCs of patients with CD compared to PBMCs of subjects substantially free of IBD, and/or in PBMCs of patients with UC compared to PBMCs of subjects substantially free of IBD. In one embodiment, PBMC- and IBD-associated biomarkers comprise the PBMC- and IBD-associated biomarkers categorized as Group I common biomarkers.

PBMC- and IBD-associated biomarkers of the invention also include Crohn's disease-associated biomarkers. As used herein, the term “Crohn's disease-associated biomarker” or “CD biomarker” includes a polynucleotide, its corresponding gene product, and fragments, homologs and isoforms thereof, that is substantially modulated (i.e., upregulated or downregulated) in quantity in peripheral blood mononuclear cells of patients with Crohn's disease compared to in PBMCs of subjects substantially free of IBD. Additionally, a CD biomarker is not substantially modulated in peripheral blood mononuclear cells of patients with ulcerative colitis compared to in PBMCs of subjects substantially free of IBD. In certain embodiments, the Crohn's disease-associated biomarkers of the invention include the PBMC- and IBD-associated biomarkers categorized as Group II biomarkers, subsets of which may be found categorized within the lists of Group IV and Group V biomarkers.

PBMC- and IBD-associated biomarkers of the invention also include ulcerative colitis-associated biomarkers. As used herein, the term “ulcerative colitis-associated biomarker” or “UC biomarker” includes a polynucleotide, its corresponding product, and fragments, homologs and isoforms thereof, that is substantially modulated (i.e., upregulated or downregulated) in quantity in peripheral blood mononuclear cells of patients with ulcerative colitis compared to PBMCs of subjects substantially free of IBD. Additionally, a UC biomarker is not substantially modulated in quantity in peripheral blood mononuclear cells of patients with Crohn's disease compared to PBMCs of subjects substantially free of IBD. In certain embodiments, the ulcerative colitis-associated biomarkers of the invention include the PBMC- and IBD-associated biomarkers categorized as Group III biomarkers, subsets of which may be found categorized within the lists of Group IV and Group V biomarkers.

PBMC- and IBD-associated biomarkers of the invention include CDvUC biomarkers. As used herein, the term “CDvUC biomarker” includes a polynucleotide, its corresponding gene product, and fragments, homologs and isoforms thereof, that is substantially modulated (i.e., upregulated or downregulated) in quantity in peripheral blood mononuclear cells of patients with either ulcerative colitis or Crohn's disease, compared to PBMCs of subjects substantially free of IBD, and that enables distinguishing peripheral blood mononuclear cells isolated from a patient with Crohn's disease from peripheral blood mononuclear cells isolated from a patient with ulcerative colitis. For example, a CDvUC biomarker may be substantially modulated in subjects with one inflammatory bowel disease, e.g., ulcerative colitis, compared to its expression in subjects with the other inflammatory bowel disease, e.g., Crohn's disease. Alternatively, a CDvUC biomarker may be modulated in opposite directions in subjects with one inflammatory bowel disease, c.g., ulcerative colitis, compared to subjects with the other inflammatory bowel disease, e.g., Crohn's disease. In certain embodiments, the distinguishing CDvUC biomarkers include the biomarkers of Group IV, a subset of which may be found categorized within the list of Group V biomarkers.

PBMC- and IBD-associated biomarkers of the invention may be categorized into smaller sets of CDvUC biomarkers, which are sets of classifying biomarkers. As used herein, “a set of classifying biomarkers” includes a set of polynucleotides, their corresponding gene products, and fragments, homologs and isoforms thereof, that may be used to distinguish patients with Crohn's disease and patients with ulcerative colitis. In certain embodiments, a set of classifying biomarkers is the set categorized as Group V biomarkers.

Preferably, for the purposes of the present invention, expression levels of the substantially modulated, i.e., upregulated or downregulated, PBMC- and IBD-associated biomarkers of the invention are respectively increased or decreased by an abnormal magnitude, wherein the level of expression is aberrant, e.g., outside the standard deviation for the same PBMC- and IBD-associated biomarker in PBMCs from healthy subjects. Most preferably, the substantially modulated PBMC- and IBD-associated biomarker is upregulated or downregulated relative to healthy subjects by at least an aberrant 1.5-, 2-, 3-, or 4-fold change or more.

The UniGene accession numbers, names of PBMC- and IBD-associated biomarkers included as Group I, Group II, Group II, Group IV, and Group V biomarkers, and the directions of their modulation (i.e., upregulation or downregulation), are listed below in Table 1, Table 2, Table 3, Table 4, and Table 5, respectively.

TABLE 1
PBMC- and IBD-associated Biomarkers of Group I; Common Biomarkers

| | | | Fold | CD vs. | Fold | UC vs. |
|---|---|---|---|---|---|---|
| | | | Difference | Normal | Difference | Normal |
| | | Direction | CD vs. | ANCOVA | UC vs. | ANCOVA |
| Accession | Name | in Both | Normal | p-value | Normal | p-value |
| Hs.75716 | serine (or cysteine) | ↑ | 6.93 | 6.84E−12 | 3.35 | 3.87E−05 |
| | proteinase inhibitor, | | | | | |
| | clade B (ovalbumin), | | | | | |
| | member 2, PAI 2 | | | | | |
| Hs.154654 | cytochrome P450, | ↑ | 3.31 | 6.49E−10 | 2.37 | 2.81E−05 |
| | subfamily I (dioxin- | | | | | |
| | inducible), | | | | | |
| | polypeptide 1 | | | | | |
| Hs.79516 | brain abundant, | ↑ | 1.94 | 6.81E−09 | 2.13 | 2.61E−09 |
| | membrane attached | | | | | |
| | signal protein 1 | | | | | |
| Hs.177781 | Unknown | ↑ | 1.88 | 6.34E−08 | 1.92 | 3.99E−07 |
| Hs.104624 | aquaporin 9 | ↑ | 1.88 | 9.92E−06 | 2.02 | 9.06E−06 |
| Hs.20084 | retinoid X receptor, | ↑ | 1.80 | 1.33E−06 | 1.68 | 9.18E−05 |
| | alpha | | | | | |
| Hs.2161 | complement | ↑ | 1.74 | 2.65E−05 | 1.81 | 5.05E−05 |
| | component 5 receptor | | | | | |
| | 1 (C5a ligand) | | | | | |
| Hs.865 | RAP1A, member of | ↑ | 1.64 | 5.95E−10 | 1.53 | 1.01E−06 |
| | RAS oncogene family | | | | | |
| Hs.177486 | amyloid beta (A4) | ↑ | 1.63 | 6.11E−10 | 1.60 | 4.81E−08 |
| | precursor protein | | | | | |
| | (protease nexin-II, | | | | | |
| | Alzheimer disease) | | | | | |
| Hs.198282 | phospholipid | ↑ | 1.60 | 3.90E−05 | 1.76 | 1.09E−05 |
| | scramblase 1 | | | | | |
| Hs.288555 | ELK3, ETS-domain | ↑ | 1.59 | 4.25E−10 | 1.51 | 2.75E−07 |
| | protein (SRF | | | | | |
| | accessory protein 2) | | | | | |
| Hs.101695 | NCK adaptor protein 2 | ↑ | 1.57 | 2.72E−14 | 1.51 | 1.08E−10 |
| Hs.198282 | phospholipid | ↑ | 1.56 | 1.02E−05 | 1.55 | 7.12E−05 |
| | scramblase 1 | | | | | |
| Hs.285313 | core promoter | ↓ | 2.78 | 8.63E−05 | 5.65 | 9.23E−09 |
| | element binding | | | | | |
| | protein | | | | | |
| Hs.151411 | KIAA0916 protein | ↓ | 2.47 | 7.92E−05 | 3.21 | 6.01E−06 |
| Hs.20072 | myosin regulatory | ↓ | 2.45 | 3.21E−07 | 2.47 | 2.73E−06 |
| | light chain interacting | | | | | |
| | protein | | | | | |
| Hs.81248 | CUG triplet repeat, | ↓ | 2.44 | 8.74E−08 | 2.52 | 4.84E−07 |
| | RNA binding protein 1 | | | | | |
| Hs.211610 | CUG triplet repeat, | ↓ | 2.27 | 5.40E−06 | 2.62 | 1.77E−06 |
| | RNA binding protein 2 | | | | | |
| Hs.86896 | bromodomain | ↓ | 2.24 | 1.22E−06 | 2.26 | 8.83E−06 |
| | containing 3 | | | | | |
| Hs.100555 | DEAD/H (Asp-Glu- | ↓ | 2.24 | 3.89E−05 | 2.48 | 3.11E−05 |
| | Ala-Asp/His) box | | | | | |
| | polypeptide 18 (Myc- | | | | | |
| | regulated) | | | | | |
| Hs.143601 | hypothetical protein | ↓ | 2.20 | 1.20E−07 | 2.16 | 2.42E−06 |
| | hCLA-iso | | | | | |
| Hs.149436 | kinesin family | ↓ | 2.15 | 2.66E−05 | 2.56 | 4.15E−06 |
| | member 5B | | | | | |
| Hs.239483 | Unknown | ↓ | 2.10 | 2.47E−09 | 2.42 | 2.47E−10 |
| Hs.78909 | zinc finger protein 36, | ↓ | 2.03 | 1.46E−07 | 2.06 | 1.18E−06 |
| | C3H type-like 2 | | | | | |
| Hs.85273 | retinoblastoma | ↓ | 2.01 | 6.23E−05 | 2.56 | 1.64E−06 |
| | binding protein 6 | | | | | |
| Hs.219614 | F-box and leucine- | ↓ | 1.95 | 9.04E−07 | 2.41 | 1.11E−08 |
| | rich repeat protein 11 | | | | | |
| Hs.153834 | pumilio homolog 1 | ↓ | 1.92 | 6.49E−08 | 1.92 | 8.29E−07 |
| | (Drosophila) | | | | | |
| Hs.18827 | cylindromatosis | ↓ | 1.91 | 7.70E−06 | 2.13 | 2.93E−06 |
| | (turban tumor | | | | | |
| | syndrome) | | | | | |
| Hs.127287 | KIAA0794 protein | ↓ | 1.83 | 3.31E−05 | 1.95 | 4.07E−05 |
| Hs.73090 | nuclear factor of | ↓ | 1.81 | 3.63E−06 | 1.77 | 4.93E−05 |
| | kappa light | | | | | |
| | polypeptide gene | | | | | |
| | enhancer in B-cells 2 | | | | | |
| | (p49/p100) | | | | | |
| Hs.373557 | partial transcript | ↓ | 1.81 | 3.43E−05 | 2.18 | 1.32E−06 |
| | encompassing | | | | | |
| | THC211630 gene | | | | | |
| Hs.75243 | Bromodomain | ↓ | 1.76 | 1.11E−06 | 1.86 | 1.65E−06 |
| | containing 2 | | | | | |
| Hs.118174 | tetratricopeptide | ↓ | 1.76 | 4.63E−05 | 2.35 | 6.54E−08 |
| | repeat domain 3 | | | | | |
| Hs.37096 | zinc finger protein | ↓ | 1.74 | 8.38E−07 | 1.91 | 2.96E−07 |
| | 145 (Kruppel-like, | | | | | |
| | expressed in | | | | | |
| | promyelocytic | | | | | |
| | leukemia) | | | | | |
| Hs.3530 | FUS interacting | ↓ | 1.72 | 2.20E−05 | 1.89 | 7.47E−06 |
| | protein (serine- | | | | | |
| | arginine rich) 1 | | | | | |
| Hs.83484 | Meis1 | ↓ | 1.70 | 2.26E−06 | 1.78 | 3.70E−06 |
| Hs.294014 | Unknown | ↓ | 1.68 | 5.43E−06 | 1.81 | 2.84E−06 |
| Hs.183418/ | cell division cycle 2- | ↓ | 1.64 | 1.42E−05 | 1.88 | 9.80E−07 |
| 214291/355896 | like 1 (PITSLRE | | | | | |
| | proteins), cell | | | | | |
| | division cycle 2-like 2 | | | | | |
| Hs.77256 | enhancer of zeste | ↓ | 1.63 | 7.72E−06 | 1.71 | 8.73E−06 |
| | homolog 2 | | | | | |
| | (Drosophila) | | | | | |
| Hs.278426 | PDGFA associated | ↓ | 1.60 | 6.59E−07 | 1.57 | 1.61E−05 |
| | protein 1 | | | | | |
| Hs.10351 | KIAA0308 protein | ↓ | 1.60 | 8.99E−06 | 1.78 | 1.10E−06 |
| Hs.18368 | SR rich protein | ↓ | 1.57 | 1.30E−05 | 1.83 | 2.43E−07 |
| Hs.2173 | fucosyltransferase 4 | ↓ | 1.54 | 4.08E−05 | 1.70 | 6.69E−06 |
| | (alpha (1,3) | | | | | |
| | fucosyltransferase, | | | | | |
| | myeloid-specific) | | | | | |
| Hs.152601 | UDP-glucose | ↓ | 1.54 | 3.35E−05 | 1.73 | 2.72E−06 |
| | ceramide | | | | | |
| | glucosyltransferase | | | | | |
| Hs.243901 | Unknown | ↓ | 1.51 | 8.84E−05 | 1.68 | 1.29E−05 |

¹These PBMC- and IBD-biomarkers have aberrant expression, e.g., are substantially upregulated (↑) or downregulated (↓), in PBMCs of both patients with CD and patients with UC compared to healthy patients.

TABLE 2
PBMC- and IBD-associated Biomarkers of Group II; CD Biomarkers

| | | | CD vs. | | |
|---|---|---|---|---|---|
| | | | Normal | CD vs. | Significant |
| | | Direction | Fold | Normal | with |
| Accession | Name | in CD | Difference | p-value | (p ≦ 0.0001) |
| Hs.72933 | platelet factor 4 variant 1 | ↑ | 2.48 | 1.91E−05 | |
| Hs.83381 | guanine nucleotide binding | ↑ | 2.30 | 4.02E−09 | |
| | protein (G protein), gamma 11 | | | | |
| Hs.119257 | emsl sequence (mammary tumor | ↑ | 2.28 | 1.53E−08 | |
| | and squamous cell carcinoma- | | | | |
| | asociated (p80/85 src substrate) | | | | |
| Hs.87149 | integrin, beta 3 (platelet | ↑ | 2.22 | 6.10E−06 | |
| | glycoprotein IIIa, antigen CD61) | | | | |
| Hs.90061 | progesterone receptor membrane | ↑ | 2.18 | 1.61E−07 | |
| | component 1 | | | | |
| Hs.155097 | carbonic anhydrase II | ↑ | 2.17 | 2.37E−06 | |
| Hs.81564 | platelet factor 4 (chemokine | ↑ | 2.14 | 4.03E−09 | |
| | (C—X—C motif) ligand 4) | | | | |
| Hs.90786 | ATP-binding cassette, sub-family | ↑ | 2.12 | 4.00E−05 | |
| | C (CFTR/MRP), member 3 | | | | |
| Hs.279843 | mutL homolog 3 (E. coli) | ↑ | 2.12 | 1.26E−08 | |
| Hs.88474 | prostaglandin-endoperoxide | ↑ | 2.06 | 4.95E−06 | |
| | synthase 1 (prostaglandin G/H | | | | |
| | synthase and cyclooxygenase) | | | | |
| Hs.75106 | clusterin (complement lysis | ↑ | 2.00 | 7.02E−06 | |
| | inhibitor, SP-40,40, sulfated | | | | |
| | glycoprotein 2, apolipoprotein J) | | | | |
| Hs.249216 | H2B histone family, member J | ↑ | 1.98 | 9.64E−08 | |
| Hs.41267 | chromosome 21 open reading | ↑ | 1.97 | 1.39E−06 | |
| | frame 7 | | | | |
| Hs.326035 | Early growth response 1 | ↑ | 1.95 | 6.86E−05 | |
| Hs.271473 | epithelial protein up-regulated in | ↑ | 1.87 | 2.30E−06 | |
| | carcinoma, membrane associated | | | | |
| | protein 17 | | | | |
| Hs.84171 | myeloproliferative leukemia virus | ↑ | 1.84 | 1.14E−06 | |
| | oncogene | | | | |
| Hs.77890 | guanylate cyclase 1, soluble, beta 3 | ↑ | 1.82 | 1.17E−06 | |
| Hs.1395 | early growth response 2 (Krox-20 | ↑ | 1.79 | 5.01E−05 | |
| | homolog, Drosophila) | | | | |
| Hs.204238 | lipocalin 2 (oncogene 24p3) | ↑ | 1.79 | 5.19E−05 | |
| Hs.88474 | prostaglandin-endoperoxide | ↑ | 1.76 | 3.21E−07 | |
| | synthase 1 (prostaglandin G/H | | | | |
| | synthase and cyclooxygenase) | | | | |
| Hs.26530 | serum deprivation response | ↑ | 1.69 | 8.41E−07 | |
| | (phosphatidylserine binding | | | | |
| | protein) | | | | |
| Hs.193700 | Consensus includes | ↑ | 1.67 | 1.12E−06 | |
| | gb: AL110164.1 | | | | |
| Hs.2164 | pro-platelet basic protein | ↑ | 1.66 | 1.18E−06 | |
| | (chemokine (C—X—C motif) | | | | |
| | ligand 7) | | | | |
| Hs.8302 | four and a half LIM domains 2 | ↑ | 1.66 | 3.70E−05 | |
| Hs.64016 | protein S (alpha) | ↑ | 1.62 | 7.63E−06 | |
| Hs.87149 | integrin, beta 3 (platelet | ↑ | 1.61 | 3.55E−05 | |
| | glycoprotein IIIa, antigen CD61) | | | | |
| Hs.114360 | transforming growth factor beta- | ↑ | 1.59 | 1.10E−06 | |
| | stimulated protein TSC-22 | | | | |
| Hs.149846 | integrin, beta 5 | ↑ | 1.58 | 1.60E−05 | |
| Hs.6721 | monoglyceride lipase | ↑ | 1.56 | 1.57E−05 | |
| Hs.77899 | tropomyosin 1 (alpha) | ↑ | 1.56 | 4.16E−05 | |
| Hs.114231 | C-type lectin-like receptor-2 | ↑ | 1.56 | 3.31E−06 | |
| Hs.7917 | likely ortholog of mouse hypoxia | ↑ | 1.55 | 1.79E−06 | |
| | induced gene 1 | | | | |
| Hs.261023 | hypothetical protein FLJ20958 | ↑ | 1.53 | 1.25E−06 | |
| Hs.22116 | CDC14 cell division cycle 14 | ↑ | 1.51 | 4.87E−05 | |
| | homolog B (S. cerevisiae) | | | | |
| Hs.433622 | follistatin-like 1 | ↑ | 1.51 | 7.19E−05 | |
| Hs.183125 | killer cell lectin-like receptor | ↓ | 2.57 | 4.03E−07 | |
| | subfamily F, member 1 | | | | |
| Hs.334837 | Williams Beuren syndrome | ↓ | 2.21 | 7.94E−06 | |
| | chromosome region 20C, | | | | |
| | Williams-Beuren Syndrome | | | | |
| | critical region protein 20 copy B | | | | |
| Hs.8272 | Prostaglandin D2 synthase 21 kDa | ↓ | 2.17 | 6.12E−08 | |
| | (brain) | | | | |
| Hs.64746 | chloride intracellular channel 3 | ↓ | 2.07 | 2.17E−05 | |
| Hs.8272 | Prostaglandin D2 synthase 21 kDa | ↓ | 2.06 | 4.36E−07 | |
| | (brain) | | | | |
| Hs.334837 | Williams Beuren syndrome | ↓ | 1.94 | 4.80E−05 | |
| | chromosome region 20A, B and C | | | | |
| Hs.406306 | Williams Beuren syndrome | ↓ | 1.88 | 4.85E−05 | |
| | chromosome region 20A, B and C | | | | |
| Hs.3066 | granzyme K (serine protease, | ↓ | 1.87 | 6.01E−05 | |
| | granzyme 3; tryptase II) | | | | |
| Hs.381613 | adaptor-related protein complex | ↓ | 1.85 | 1.06E−05 | |
| | 1, gamma 2 subunit | | | | |
| Hs.355888 | phospholipase C, beta 2 | ↓ | 1.83 | 7.22E−06 | |
| Hs.88411 | natural cytotoxicity triggering | ↓ | 1.82 | 1.22E−05 | |
| | receptor 3 | | | | |
| Hs.406306 | Williams Beuren syndrome | ↓ | 1.80 | 3.32E−05 | |
| | chromosome region 20A, B and C | | | | |
| Hs.194669 | enhancer of zeste homolog 1 | ↓ | 1.80 | 5.58E−05 | |
| | (Drosophila) | | | | |
| Hs.99491 | RAS guanyl releasing protein 2 | ↓ | 1.77 | 5.81E−07 | |
| | (calcium and DAG-regulated | | | | |
| Hs.349256 | paired immunoglobulin-like | ↓ | 1.73 | 9.12E−05 | |
| | receptor beta | | | | |
| Hs.13377 | abhydrolase domain containing 3 | ↓ | 1.70 | 5.09E−05 | neutrophils |
| Hs.274 | megakaryocyte-associated | ↓ | 1.68 | 3.10E−06 | monocytes/ |
| | tyrosine kinase | | | | lymphocytes |
| Hs.323817 | DKFZP547E1010 protein | ↓ | 1.66 | 2.68E−06 | |
| Hs.8272 | prostaglandin D2 synthase 21 kDa | ↓ | 1.64 | 7.56E−06 | |
| | (brain) | | | | |
| Hs.288126 | spondin 2, extracellular matrix | ↓ | 1.64 | 6.34E−06 | |
| | protein | | | | |
| Hs.8182 | hypothetical protein MGC17528, | ↓ | 1.63 | 1.44E−07 | |
| | synaptic nuclei expressed gene 1 | | | | |
| Hs.31834 | Consensus includes gb: AI052536 | ↓ | 1.63 | 2.88E−05 | |
| Hs.234569 | zeta-chain (TCR) associated | ↓ | 1.61 | 3.24E−05 | monocytes/ |
| | protein kinase 70 kDa | | | | lymphocytes |
| Hs.167988 | neural cell adhesion molecule 1 | ↓ | 1.61 | 2.19E−05 | |
| Hs.75196 | HLA-B associated transcript 8 | ↓ | 1.61 | 6.42E−05 | |
| Hs.180948 | KIAA0570 gene product | ↓ | 1.60 | 4.03E−05 | |
| Hs.356684 | hypothetical protein | ↓ | 1.58 | 3.27E−06 | eosinophils & |
| | DKFZp762C186 | | | | neutrophils |
| Hs.29288 | endo-beta-N- | ↓ | 1.55 | 1.69E−05 | |
| | acetylglucosaminidase | | | | |
| Hs.313844 | Consensus includes | ↓ | 1.55 | 8.69E−05 | |
| | gb: AW069290 | | | | |
| Hs.100293 | O-linked N-acetylglucosamine | ↓ | 1.53 | 1.41E−05 | |
| | (GlcNAc) transferase (UDP-N- | | | | |
| | acetylglucosamine:polypeptide- | | | | |
| | N-acetylglucosaminyl transferase) | | | | |
| unknown | Consensus includes | ↓ | 1.52 | 6.06E−07 | |
| | gb: NM_024957.1 | | | | |
| Hs.170160 | RAB2, member RAS oncogene | ↓ | 1.52 | 2.47E−06 | neutrophils |
| | family-like | | | | |

²These PBMC- and IBD-biomarkers have aberrant expression, e.g., are substantially upregulated (↑) or downregulated (↓), only in PBMCs of patients with CD (i.e., not in PBMCs of patients with UC) compared to healthy patients.

TABLE 3
PBMC- and IBD-associated Biomarkers of Group III; UC Biomarkers

| | | | UC vs. | | |
|---|---|---|---|---|---|
| | | | Normal | UC vs. | Significant |
| | | Direction | Fold | Normal | with |
| Accession | Name | in UC | Difference | p-value | (p ≦ 0.0001) |
| Hs.300697 | immunoglobulin heavy constant | ↑ | 4.65 | 2.70E−12 | |
| | gamma 3 (G3m marker) | | | | |
| N/A | Consensus includes gb: X51887 | ↑ | 2.75 | 7.83E−05 | |
| | (immunoglobulin kappa orphon) | | | | |
| Hs.406565 | immunoglobulin kappa constant | ↑ | 2.63 | 2.05E−06 | |
| Hs.76325 | immunoglobulin J polypeptide, | ↑ | 2.48 | 4.70E−06 | |
| | linker protein for immunoglobulin | | | | |
| | alpha and mu polypeptides | | | | |
| Hs.406565 | immunoglobulin kappa constant | ↑ | 2.46 | 4.66E−08 | |
| Hs.102950 | Coatomer protein complex, | ↑ | 2.32 | 3.12E−06 | |
| | subunit gamma, immunoglobulin | | | | |
| | lambda joining 3 | | | | |
| Hs.406565 | immunoglobulin kappa constant | ↑ | 2.21 | 5.90E−10 | |
| Hs.102950 | Coatomer protein complex, | ↑ | 2.19 | 3.33E−07 | |
| | subunit gamma, immunoglobulin | | | | |
| | lambda joining 3 | | | | |
| N/a | Consensus includes gb: AJ408433 | ↑ | 2.16 | 1.92E−05 | |
| | (partial IGKV gene for | | | | |
| | immunoglobulin kappa chain | | | | |
| | variable region) | | | | |
| Hs.406565 | immunoglobulin kappa constant | ↑ | 2.16 | 7.59E−07 | |
| Hs.102950 | Coatomer protein complex, | ↑ | 2.10 | 4.74E−06 | |
| | subunit gamma, immunoglobulin | | | | |
| | lambda joining 3 | | | | |
| Hs.406565 | immunoglobulin kappa constant | ↑ | 2.05 | 1.99E−06 | |
| Hs.381417 | Consensus includes | ↑ | 1.97 | 3.63E−06 | |
| | gb: AF103529.1 (immunoglobulin | | | | |
| | kappa light chain variable region) | | | | |
| Hs.348935 | immunoglobulin lambda-like | ↑ | 1.89 | 1.65E−06 | |
| | polypeptide 1 | | | | |
| Hs.405944 | immunoglobulin lambda locus | ↑ | 1.83 | 2.53E−05 | |
| Hs.102950 | Coatomer protein complex, | ↑ | 1.82 | 1.55E−05 | |
| | subunit gamma, immunoglobulin | | | | |
| | lambda joining 3 | | | | |
| Hs.405944 | immunoglobulin lambda locus | ↑ | 1.75 | 7.51E−06 | |
| Hs.406565 | immunoglobulin kappa constant | ↑ | 1.71 | 1.12E−07 | |
| Hs.381418 | Consensus includes | ↑ | 1.70 | 3.11E−07 | |
| | gb: AF103530.1(immunoglobulin | | | | |
| | kappa light chain variable region) | | | | |
| Hs.406565 | immunoglobulin kappa constant | ↑ | 1.59 | 4.25E−06 | |
| Hs.102950 | Coatomer protein complex, | ↑ | 1.50 | 2.91E−05 | |
| | subunit gamma, immunoglobulin | | | | |
| | lambda joining 3 | | | | |
| Hs.107213 | formin binding protein 3 | ↓ | 1.75 | 4.27E−05 | |

³These PBMC- and IBD-biomarkers have aberrant expression, e.g., are substantially upregulated (↑) or downregulated (↓), only in PBMCs of patients with UC (i.e., not in PBMCs of patients with CD) compared to healthy patients.

TABLE 4
PBMC- and IBD-associated Biomarkers of Group IV; CDvUC Biomarkers

| | | | Fold | ANCOVA |
|---|---|---|---|---|
| Accession | Name | Specific to: | Difference | p-value |
| Hs.90061 | progesterone receptor membrane | Crohn's | 2.08 | 2.55E−07 |
| | component 1 | | | |
| Hs.279843 | mutL homolog 3 (E. coli) | Crohn's | 2.00 | 2.75E−08 |
| Hs.88474 | prostaglandin-endoperoxide synthase 1 | Crohn's | 1.93 | 1.18E−05 |
| | (prostaglandin G/H synthase and | | | |
| | cyclooxygenase) | | | |
| Hs.73769 | folate receptor 1 (adult) | Crohn's | 1.93 | 6.82E−11 |
| Hs.89714 | chemokine (C—X—C motif) ligand 5 | Crohn's | 1.85 | 3.71E−05 |
| Hs.83381 | guanine nucleotide binding protein (G | Crohn's | 1.79 | 8.04E−06 |
| | protein), gamma 11 | | | |
| Hs.2359 | dual specificity phosphatase 4 | Crohn's | 1.76 | 5.30E−05 |
| Hs.204238 | lipocalin 2 (oncogene 24p3) | Crohn's | 1.75 | 4.35E−05 |
| Hs.81564 | platelet factor 4 (chemokine (C—X—C | Crohn's | 1.74 | 4.38E−06 |
| | motif) ligand 4) | | | |
| Hs.119257 | ems1 sequence (mammary tumor and | Crohn's | 1.70 | 8.21E−05 |
| | squamous cell carcinoma-associated | | | |
| | (p80/85 src substrate) | | | |
| Hs.26530 | serum deprivation response | Crohn's | 1.66 | 5.34E−07 |
| | (phosphatidylserine binding protein) | | | |
| Hs.303023 | tubulin, beta 1 | Crohn's | 1.65 | 8.39E−05 |
| Hs.23581 | leptin receptor gene-related protein | Crohn's | 1.61 | 7.52E−05 |
| Hs.249216 | H2B histone family, member J | Crohn's | 1.61 | 6.91E−05 |
| Hs.77439 | protein kinase, cAMP-dependent, | Crohn's | 1.59 | 4.09E−05 |
| | regulatory, type II, beta | | | |
| Hs.2178 | H2B histone family, member Q | Crohn's | 1.57 | 8.85E−06 |
| Hs.114231 | C-type lectin-like receptor-2 | Crohn's | 1.57 | 8.79E−07 |
| Hs.12813 | DKFZP434J214 protein | Crohn's | 1.52 | 1.21E−05 |
| Hs.2164 | pro-platelet basic protein (chemokine | Crohn's | 1.51 | 2.90E−05 |
| | (C—X—C motif) ligand 7) | | | |
| Hs.300697 | immunoglobulin heavy constant gamma 3 | UC | 3.87 | 9.28E−13 |
| | (G3m marker) | | | |
| Hs.153261 | immunoglobulin heavy constant mu | UC | 2.60 | 2.72E−05 |
| n/a | unknown EST with consensus to | UC | 2.42 | 5.66E−05 |
| | immunoglobulin kappa orphon | | | |
| Hs.406565 | immunoglobulin kappa constant | UC | 2.30 | 1.78E−06 |
| n/a | 28 S ribosomal RNA 5′ region | UC | 2.11 | 2.95E−07 |
| Hs.406565 | immunoglobulin kappa constant | UC | 2.08 | 2.88E−08 |
| Hs.406565 | immunoglobulin kappa constant | UC | 2.04 | 3.52E−07 |
| Hs.183125 | killer cell lectin-like receptor subfamily F, | UC | 2.02 | 5.45E−05 |
| | member 1 | | | |
| Hs.411106 | perforin 1 | UC | 1.98 | 7.07E−05 |
| Hs.153261 | immunoglobulin heavy constant mu | UC | 1.93 | 3.78E−05 |
| Hs.406565 | immunoglobulin kappa constant | UC | 1.88 | 2.19E−06 |
| Hs.406565 | immunoglobulin kappa constant | UC | 1.87 | 8.32E−09 |
| Hs.102950 | coatomer protein complex, subunit gamma, | UC | 1.74 | 5.47E−05 |
| | immunoglobulin lambda joining 3 | | | |
| Hs.102950 | coatomer protein complex, subunit gamma, | UC | 1.74 | 2.10E−05 |
| | immunoglobulin lambda joining 3 | | | |
| Hs.406565 | immunoglobulin kappa constant | UC | 1.72 | 2.57E−07 |
| Hs.355888 | phospholipase C, beta 2 | UC | 1.72 | 2.24E−05 |
| Hs.8272 | prostaglandin D2 synthase 21 kDa (brain) | UC | 1.71 | 5.41E−05 |
| Hs.25338 | protease, serine, 23 | UC | 1.68 | 7.72E−05 |
| Hs.381417 | unknown EST with consensus to | UC | 1.67 | 3.86E−05 |
| | immunoglobulin kappa light chain variable | | | |
| | region | | | |
| Hs.75596 | interleukin 2 receptor, beta | UC | 1.65 | 7.19E−05 |
| Hs.406565 | immunoglobulin kappa constant | UC | 1.64 | 2.48E−06 |
| Hs.102950 | coatomer protein complex, subunit gamma, | UC | 1.64 | 4.13E−05 |
| | immunoglobulin lambda joining 3 | | | |
| Hs.406565 | immunoglobulin kappa constant | UC | 1.63 | 4.95E−06 |
| Hs.406565 | immunoglobulin kappa constant | UC | 1.61 | 3.75E−08 |
| Hs.380156 | NK-receptor, killer cell immunoglobulin- | UC | 1.60 | 3.16E−08 |
| | like receptor, two domains, long | | | |
| | cytoplasmic tail, 3 | | | |
| Hs.405944 | immunoglobulin lambda locus | UC | 1.59 | 1.55E−05 |
| Hs.348935 | immunoglobulin lambda-like polypeptide 1 | UC | 1.58 | 4.69E−05 |
| Hs.84 | interleukin 2 receptor, gamma (severe | UC | 1.53 | 7.84E−05 |
| | combined immunodeficiency) | | | |
| Hs.193128 | homolog of C, elegans smu-1 | UC | 1.52 | 5.16E−05 |
| Hs.238944 | hypothetical protein FLJ10631 | UC | 1.52 | 3.72E−05 |

TABLE 5
PBMC- and IBD-associated Biomarkers of Group V; Classifying Biomarkers

| Classifier | | | Unigene |
|---|---|---|---|
| Gene | Class | Name | ID |
| 1 | Crohn's | Lipocalin 2 (oncogene 24p3) | Hs.204238 |
| 2 | Crohn's | mutL homolog 3 (E. coli) | Hs.279843 |
| 3 | Crohn's | serum deprivation response (phosphatidylserine binding protein) | Hs.26530 |
| 4 | Crohn's | H2B histone family, member Q | Hs.2178 |
| 5 | Crohn's | H3 histone family, member K | Hs.70937 |
| 6 | Crohn's | chemokine (C—X—C motif) ligand 5 | Hs.89714 |
| 7 | Crohn's | integrin, beta 3 (platelet glycoprotein IIIa, antigen CD61) | Hs.87149 |
| 8 | UC | immunoglobulin heavy constant gamma 3 (G3m marker; IgHg3) | Hs.300697 |
| | | also referred to herein as immunoglobulin heavy constant | |
| | | gamma 1 | |
| 9 | UC | immunoglobulin kappa constant | Hs.406565 |
| 10 | UC | M27830 Human 28S ribosomal RNA gene 5′ region | n/a |
| 11 | UC | protein tyrosine phosphatase, receptor type, C-associated protein | Hs.155975 |
| 12 | UC | granzyme K (serine protease, granzyme 3; tryptase II) | Hs.3066 |
| 13 | UC | immunoglobulin kappa constant | Hs.406565 |
| 14 | UC | immunoglobulin kappa constant | Hs.406565 |

Sources of PBMC- and IBD-associated Biomarkers

The polynucleotide and polypeptide of a PBMC- and IBD-associated ker of the invention may be isolated from any tissue or cell of a subject expressing the PBMC- and IBD-associated biomarker. In a preferred, nonlimiting embodiment, the tissue is from blood (or, e.g., serum, plasma, blood cells), lymph nodes, saliva, stomach, or intestine. The tissue samples containing one or more of the PBMC- and IBD-associated biomarkers themselves may be useful in the methods of the invention, and one skilled in the art will be cognizant of the methods by which such samples may be conveniently obtained, stored and or preserved. However, it will be apparent to one skilled in the art that blood, in particular, PBMCs, would serve as a preferred source from which the expression of PBMC- and IBD-associated biomarkers of the invention are assessed in the provided methods of diagnosing, prognosing, and/or monitoring gress of an IBD, i.e., CD or UC.

Isolated PBMC- and IBD-associated Biomarker Polynucleotides

The present invention provides isolated polynucleotides and polypeptides as PBMC- and IBD-associated biomarkers. Preferred nucleotide sequences of the invetion include genomic, cDNA, mRNA, siRNA, and chemically synthesized nucleotide sequences.

Exemplary PBMC- and IBD-associated biomarkers of the invention are listed in Tables 1-5. The invention encompasses the polynucleotide sequences of the PBMC- and IBD-associated biomarkers listed in Tables 1-5. Polynucleotides of the present invention also include polynucleotides that hybridize under stringent conditions to the polynucleotides sequences of the PBMC- and IBD-associated biomarkers listed in Tables 1-5, or their complements, and/or encode polypeptides that retain substantial biological activity (i.e., active fragments) of the PBMC- and IBD-associated biomarkers listed in Tables 1-5. Polynucleotides of the present invention also include continuous portions of the polynucleotide sequences of the PBMC- and IBD-associated biomarkers listed in Tables 1-5 comprising at least 21 consecutive nucleotides.

The invention further encompasses the polypeptides of the PBMC- and IBD-associated biomarkers listed in Tables 1-5. Polypeptides of the present invention also include continuous portions of the polypeptides of the PBMC- and IBD-associated biomarkers listed in Tables 1-5 comprising at least 7 consecutive amino acids. A preferred embodiment of the invention includes any continuous portion of any of the polypeptides of the PBMC- and IBD-associated biomarkers selected from those listed in Tables 1-5 that retains substantial biological activity of the selected polypeptide.

The invention further encompasses polynucleotide molecules that differ from the polynucleotide sequences of the PBMC- and IBD-associated biomarkers listed in Tables 1-5 only due to the well-known degeneracy of the genetic code, and which thus encode the same proteins as those encoded by the PBMC- and IBD-associated biomarkers listed in Tables 1-5.

The polynucleotides encompassed by the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridization, in situ hybridization, and Northern hybridization, and are well known to those skilled in the art.

Hybridization reactions can be performed under conditions of different stringency. The stringency of a hybridization reaction includes the difficulty with which any two nucleic acid molecules will hybridize to one another. The present invention also includes polynucleotides capable of hybridizing under reduced stringency conditions, more preferably stringent conditions, and most preferably highly stringent conditions, to polynucleotides described herein. Examples of stringency conditions are shown in Table 6 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 6
Stringency Conditions

| | | Hybrid | | Wash |
|---|---|---|---|---|
| Stringency | Polynucleotide | Length | Hybridization Temperature and | Temperature and |
| Condition | Hybrid | (bp)¹ | Buffer² | Buffer² |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should | | | | |
| # be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(% G + C) − (600/N), | | | | |
| # where N is the number of bases in the hybrid, and Na⁺ is the concentration of sodium ions in the hybridization buffer (Na⁺ for 1xSSC = 0.165M). | | | | |
| Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, | | | | |
| # 1995, F. M. Ausubel et al., eds., John Wiley & Sons, Inc., sections 2.10 and 6.3-6.4, incorporated herein by reference. | | | | |

The polynucleotides of the present invention may also be used as hybridization probes and primers to identify and isolate homologous polynucleotides, i.e., nucleic acids having sequences that encode polypeptides of the invention and/or polypeptides homologous to the disclosed polypeptides. These homologs are polynucleotides and polypeptides isolated from different species than that of the disclosed polynucleotides and polypeptides, or within the same species, but with significant sequence similarity to the disclosed polynucleotides and polypeptides. Preferably, polynucleotide homologs have at least 60% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species, most preferably those isolated from humans.

The polynucleotides of the present invention may be used as hybridization probes and primers to identify and isolate DNAs having sequences encoding allelic variants of the polynucleotides sequences of the PBMC- and IBD-associated biomarkers listed in Tables 1-5. Allelic variants are naturally occurring alternative forms of the polynucleotide sequences of the PBMC- and IBD-associated biomarkers listed in Tables 1-5 that encode polypeptides that are identical to or have significant similarity to the polypeptides encoded by the genes listed in Tables 1-5. Preferably, allelic variants have at least 90% sequence identity (more preferably, at least 95% identity; most preferably, at least 99% identity) with the disclosed polynucleotides.

Consequently, in addition to polynucleotide sequences listed in Tables 1-5, the present invention also encompasses homologs and allelic variants of the PBMC- and IBD-associated biomarkers listed in Tables 1-5.

The polynucleotides of the present invention may also be used as hybridization probes and primers to identify cells and tissues that express the polypeptides of PBMC- and IBD-associated biomarkers of the present invention and the conditions under which they are expressed.

Additionally, the polynucleotides of the present invention may be used to alter (i.e., regulate (e.g., enhance, reduce, or modify)) the expression of the genes corresponding to the PBMC- and IBD-associated biomarkers of the present invention in a cell or organism. These corresponding genes are the genomic DNA sequences of the present invention that are transcribed to produce the mRNAs from which the PBMC- and IBD-associated biomarker polypeptides of the present invention are derived.

Altered expression of the PBMC- and IBD-associated biomarkers encompassed by the present invention in a cell or organism may be achieved through the use of various inhibitory polynucleotides, such as antisense polynucleotides, ribozymes that bind and/or cleave the mRNA transcribed from the genes of the invention, triplex-forming oligonucleotides that target regulatory regions of the genes, and short interfering RNA that causes sequence-specific degradation of target mRNA (e.g., Galderisi et al. (1999) J. Cell. Physiol. 181:251-57; Sioud (2001) Curr. Mol. Med 1:575-88; Knauert and Glazer (2001) Hum. Mol. Genet. 10:2243-51; Bass (2001) Nature 411:428-29).

The inhibitory antisense or ribozyme polynucleotides of the invention can be complementary to an entire coding strand of a gene of the invention, or to only a portion thereof. Alternatively, inhibitory polynucleotides can be complementary to a noncoding region of the coding strand of a gene of the invention. The inhibitory polynucleotides of the invention can be constructed using chemical synthesis and/or enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Mickelfield (2001) Curr. Med. Chem. 8:1157-79). Alternatively, antisense molecules can be produced biologically using an expression vector into which a polynucleotide of the present invention has been subcloned in an antisense (i.e., reverse) orientation.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2′-o-methylribonucleotide or a chimeric RNA-DNA analogue, according to techniques that are known in the art.

The inhibitory triplex-forming oligonucleotides (TFOs) encompassed by the present invention bind in the major groove of duplex DNA with high specificity and affinity (Knauert and Glazer, supra). Expression of the genes of the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nature Med. 9:347-51). In addition, intrathecally delivered siRNA has recently been reported to block pain responses in two models (agonist-induced pain model and neuropathic pain model) in the rat (Dom et al. (2004) Nucleic Acids Res. 32(5):e49).

The siRNA molecules of the present invention can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad Sci. USA 99:6047-52). The siRNA molecules can be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to the polynucleotides of the present invention can be designed based on criteria well known in the art (e.g., Elbashir et al. (2001) EMBO J 20:6877-88). For example, the target segment of the target mRNA should begin with AA (preferred), TA, GA, or CA; the GC ratio of the siRNA molecule should be 45-55%; the siRNA molecule should not contain three of the same nucleotides in a row; the siRNA molecule should not contain seven mixed G/Cs in a row; and the target segment should be in the ORF region of the target MRNA and should be at least 75 bp after the initiation ATG and at least 75 bp before the stop codon. siRNA molecules targeted to the polynucleotides of the present invention can be designed by one of ordinary skill in the art using the aforementioned criteria or other known criteria (e.g., Reynolds et al. (2004) Nat. Biotechnol. 22:326-30).

Altered expression of the genes of PBMC- and IBD-associated biomarkers of the present invention in a cell or organism may also be achieved through the creation of nonhuman transgenic animals into whose genomes polynucleotides of the present invention have been introduced. Such transgenic animals include animals that have multiple copies of a gene (i.e., the transgene) of the present invention. A tissue-specific regulatory sequence(s) may be operably linked to the transgene to direct expression of a polypeptide of the present invention to particular cells or a particular developmental stage. In another embodiment, transgenic nonhuman animals can be produced that contain selected systems that allow for regulated expression of the transgene. One example of such a system known in the art is the cre/loxP recombinase system of bacteriophage P1. Methods for generating transgenic animal via embryo manipulation and microinjection, particularly animals such as mice, have become conventional and are well known in the art (e.g., Bockamp et al. (2002) Physiol. Genomics 11:115-32). In preferred embodiments of the invention, the nonhuman transgenic animal comprises at least one PBMC- and IBD-associated biomarker.

Altered expression of the genes of the present invention in a cell or organism may also be achieved through the creation of animals whose endogenous genes corresponding to the polynucleotides of the present invention have been disrupted through insertion of extraneous polynucleotides sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-finctional protein. Methods for generating knockout animals include homologous recombination and are well known in the art (e.g., Wolfer et al. (2002) Trends Neurosci. 25:336-40).

Isolated PBMC- and IBD-associated Biomarker Polypeptides

Several aspects of the invention pertain to isolated PBMC- and IBD-associated biomarker proteins, biologically active portions thereof, and polypeptide fragments suitable for use as immunogens to raise anti- PBMC- and IBD-associated biomarker antibodies. In one embodiment, native PBMC- and IBD-associated biomarker proteins can be isolated from cells or tissue sources by an appropriate purification scheme using standard protein purification techniques. In another embodiment, PBMC- and IBD-associated biomarker proteins are produced by recombinant DNA techniques. As an alternative to recombinant expression, a PBMC- and IBD-associated biomarker protein or polypeptide can be synthesized chemically using standard peptide synthesis techniques.

The PBMC- and IBD-associated biomarker proteins listed in Tables 1-5 may be recombinantly produced by operably linking the polynucleotide sequences of the PBMC- and IBD-associated biomarkers listed in Tables 1-5 to an expression control sequence (e.g., the pMT2 and pED expression vectors). General methods of expressing recombinant proteins are well known in the art.

A number of cell lines may act as suitable host cells for recombinant expression of PBMC- and IBD-associated biomarker polypeptides of the present invention. Mammalian host cell lines include, for example, COS cells, CHO cells, 293T cells, A431 cells, 3T3 cells, CV-1 cells, HeLa cells, L cells, BHK21 cells, HL-60 cells, U937 cells, HaK cells, Jurkat cells, normal diploid cells, as well as cell strains derived from in vitro culture of primary tissue and primary explants.

Alternatively, it may be possible to recombinantly produce the polypeptides of the present invention in lower eukaryotes, such as yeast, or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides of the present invention are made in yeast or bacteria, it may be necessary to modify them by, for example, phosphorylation or glycosylation of appropriate sites in order to obtain finctionality. Such covalent attachments may be accomplished using well-known chemical or enzymatic methods.

In another embodiment of the invention, PBMC- and IBD-associated biomarker polypeptides of the present invention may also be recombinantly produced by operably linking the PBMC- and IBD-associated biomarker polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MAXBAC® kit, Invitrogen, Carlsbad, Calif.).

Following recombinant expression in the appropriate host cell, the polypeptides of the present invention may then be purified from culture medium or cell extracts using well-known purification processes, such as gel filtration and ion exchange chromatography. Purification may also include affinity chromatography with agents known to bind the polypeptides of the present invention. These purification processes may also be used to purify the polypeptides of the present invention from natural sources.

Alternatively, the PBMC- and IBD-associated biomarker polypeptides of the present invention may also be expressed recombinantly in a form that facilitates identification, purification and/or detection. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen (Carlsbad, Calif.), respectively. The polypeptides of the present invention can also be tagged with a small epitope and subsequently identified or purified using a specific antibody to the epitope. A preferred epitope is the FLAG epitope, which is commercially available from Eastman Kodak (New Haven, Conn).

A signal sequence can be used to facilitate secretion and isolation of the secreted protein or other proteins of interest. Signal sequences are typically characterized by a core of hydrophobic amino acids that are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein that is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art-recognized methods. Alternatively, the signal sequence can be linked to the protein of interest using a sequence that facilitates purification, such as with a GST domain.

In addition to the PBMC- and IBD-associated biomarker polypeptides listed in Tables 1-5, and allelic variants and homologs thereof, the present invention also encompasses polypeptides that are structurally different from the polypeptides listed in Tables 1-5 (e.g., have a slightly altered sequence), but that have substantially the same biochemical properties as the disclosed polypeptides (e.g., are changed only in functionally nonessential amino acid residues). Such molecules include, but are not limited to, deliberately engineered variants containing alterations, substitutions, replacements, insertions, or deletions. Techniques and kits for such alterations, substitutions, replacements, insertions or deletions are well known to those skilled in the art.

The present invention also encompasses variants of the PBMC- and IBD-associated biomarker proteins of the invention that function either as agonists or as antagonists to the PBMC- and IBD-associated biomarker proteins. In certain embodiments, an agonist of the PBMC- and IBD-associated biomarker proteins can retain substantially the same, or a subset of, the biological activities of the naturally occurring form of a PBMC- and IBD-associated biomarker protein, or may enhance an activity of a PBMC- and IBD-associated biomarker protein. In certain embodiments, an antagonist of a PBMC- and IBD-associated biomarker protein can inhibit one or more of the activities of the naturally occurring form of the PBMC- and IBD-associated biomarker protein by, for example, competitively modulating an activity of a PBMC- and IBD-associated biomarker protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the PBMC- and IBD-associated biomarker protein. In another preferred embodiment, an agent may serve as an agonist or an antagonist for PBMC- and IBD-associated biomarker proteins of the invention depending on whether up- or downregulation of a particular PBMC- and IBD-associated biomarker protein of interest is required for treatment of IBD.

Variants of the PBMC- and IBD-associated biomarker proteins can be generated by mutagenesis, e.g., discrete point mutation or truncation of a PBMC- and IBD-associated biomarker protein. Alternatively, variants of PBMC- and IBD-associated biomarker proteins that function as either PBMC- and IBD-associated biomarker protein agonists or as PBMC- and IBD-associated biomarker protein antagonists can be identified by screening combinatorial libraries of mutants, e.g., truncation mutants of a PBMC- and IBD-associated biomarker protein for agonist or antagonist activity. In one embodiment, a variegated library of PBMC- and IBD-associated biomarker protein variants is generated by combinatorial mutagenesis at the polynucleotide level and is encoded by a variegated gene library. In certain embodiments, such protein may be used, for example, as a therapeutic protein of the invention. A variegated library of PBMC- and IBD-associated biomarker protein variants can be produced by, for example, enzymatically ligating a mixture of synthetic oligonucleotides into gene sequences such that a degenerate set of potential PBMC- and IBD-associated biomarker protein sequences is expressible as individual polypeptides, or alternatively, as a set of larger fusion proteins (e.g., for phage display) containing the set of PBMC- and IBD-associated biomarker protein sequences therein. There are a variety of methods that can be used to produce libraries of potential PBMC- and IBD-associated biomarker protein variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PBMC- and IBD-associated biomarker protein sequences. Methods for synthesizing degenerate oligonucleotides are known in the art.

The polypeptides of the present invention may also be produced by known conventional chemical synthesis. Methods for chemically synthesizing the polypeptides of the present invention are well known to those skilled in the art. Such chemically synthetic polypeptides may possess biological properties in common with the natural, purified polypeptides, and thus may be employed as biologically active or immunological substitutes for the natural peptides.

Antibodies Against PBMC- and IBD-associated Biomarkers

In another aspect, the invention pertains to antibodies that are specific to proteins corresponding to, or encoded by, PBMC- and IBD-associated biomarkers of the invention. Preferably the antibodies are monoclonal, and most preferably, the antibodies are humanized, as described below.

Antibody molecules to the PBMC- and IBD-associated biomarkers of the invention (anti-biomarker antibodies) may be produced by methods well known to those skilled in the art. For example, monoclonal antibodies can be produced by generation of hybridomas in accordance with known methods. Hybridomas formed in this manner are then screened using standard methods, such as enzyme-linked immunosorbent assay (ELISA), to identify one or more hybridomas that produce an antibody that specifically binds with the polypeptides of the present invention. A full-length polypeptide of the present invention may be used as the immunogen, or, alternatively, antigenic peptide fragments of the polypeptides may be used. An antigenic peptide of a polypeptide of the present invention comprises at least 7 continuous amino acid residues, and encompasses an epitope such that an antibody raised against the peptide forms a specific immune complex with the polypeptide. Preferably, the antigenic peptide comprises at least 10 amino acid residues, more preferably at least 15 amino acid residues, even more preferably at least 20 amino acid residues, and most preferably at least 30 amino acid residues.

As an alternative to preparing monoclonal antibody-secreting hybridomas, a monoclonal antibody to a PBMC- and IBD-associated biomarker of the present invention may be identified and isolated by screening a recombinant combinatorial immunoglobulin library (e.g., an antibody phage display library) with a PBMC- and IBD-associated biomarker polypeptide of the present invention to thereby isolate immunoglobulin library members that bind to the PBMC- and IBD-associated biomarker. Techniques and commercially available kits for generating and screening phage display libraries are well known to those skilled in the art, as are methods and reagents particularly amenable for use in generating and screening antibody display libraries.

Polyclonal sera and antibodies may be produced by immunizing a suitable subject with a polypeptide of the present invention. The antibody titer in the immunized subject may be monitored over time by standard techniques, such as with ELISA using immobilized biomarker protein. If desired, the antibody molecules directed against a polypeptide of the present invention may be isolated from the subject or culture media and further purified by well-known techniques, such as protein A chromatography, to obtain an IgG fraction.

Additionally, recombinant anti-biomarker antibodies, such as chimeric, humanized, and single-chain antibodies, comprising both human and nonhuman portions, which can be made using standard recombinant DNA techniques, are within the scope of the invention. Humanized antibodies may also be produced using transgenic mice that are incapable of expressing endogenous immunoglobulin heavy and light chain genes, but that can express human heavy and light chain genes. Alternatively, humanized antibodies that recognize a selected epitope can be generated using a technique referred to as guided selection. In this approach, a selected nonhuman monoclonal antibody (e.g., a murine antibody) is used to guide the selection of a humanized antibody recognizing the same epitope.

Chimeric antibodies, including chimeric immunoglobulin chains, may be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see PCT/US86/02269; EP 184,187; EP 171,496; EP 173,494; WO 86/01533; U.S. Pat. No. 4,816,567; EP 125,023; Better et al. (1988) Science 240:1041-43; Liu et al. (1987) Proc. Natl. Acad. Sci. U.S.A. 84:3439-43; Liu et al. (1987) J Immunol. 139:3521-26; Sun et al. (1987) Proc. Natl. Acad Sci. U.S.A. 84:214-18; Nishimura et al. (1987) Canc. Res. 47:999-1005; Wood et al. (1985) Nature 314:446-49; and Shaw et al. (1988) J. Natl. Cancer Inst. 80:1553-59).

If desired, an antibody or an immunoglobulin chain may be humanized by methods known in the art. Humanized antibodies, including humanized immunoglobulin chains, may be generated by replacing sequences of the Fv variable region that are not directly involved in antigen binding with equivalent sequences from human Fv variable regions. General methods for generating humanized antibodies are provided by Morrison (1985) Science 229:1202-07; Oi et al. (1986) BioTechniques 4:214-21; and U.S. Pat. Nos. 5,585,089, 5,693,761 and 5,693,762, all of which are hereby incorporated by reference in their entireties. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target. The recombinant DNA encoding the humanized antibody, or fragment thereof, may then be cloned into an appropriate expression vector.

Humanized or CDR-grafted antibody molecules or immunoglobulins may be produced by CDR grafting or CDR substitution, wherein one, two, or all CDRs of an immunoglobulin chain can be replaced. See, e.g., U.S. Pat. No. 5,225,539; Jones et al. (1986) Nature 321:522-25; Verhoeyan et al. (1988) Science 239:1534-36; and Beidler et al. (1988) J. Immunol. 141:4053-60, all of which are hereby incorporated by reference in their entireties. U.S. Pat. No. 5,225,539 describes a CDR-grafting method that may be used to prepare humanized antibodies of the present invention (see also, GB 2188638A). All of the CDRs of a particular human antibody may be replaced with at least a portion of a nonhuman CDR, or only some of the CDRs may be replaced with nonhuman CDRs. It is only necessary to replace the number of CDRs required for binding of the humanized antibody to a predetermined antigen.

Monoclonal, chimeric and humanized antibodies, which have been modified by, e.g., deleting, adding, or substituting other portions of the antibody, e.g., the constant region, are also within the scope of the invention. For example, an antibody may be modified as follows: (i) by deleting the constant region; (ii) by replacing the constant region with another constant region, e.g., a constant region meant to increase half-life, stability or affinity of the antibody, or a constant region from another species or antibody class; and/or (iii) by modifying one or more amino acids in the constant region to alter, for example, the number of glycosylation sites, effector cell function, Fc receptor (FcR) binding, complement fixation, among others.

Methods for altering an antibody constant region are known in the art. Antibodies with altered fuiction (e.g., altered affinity for an effector ligand, such as FcR on a cell, or the C1 component of complement) may be produced by replacing at least one amino acid residue in the constant portion of the antibody with a different residue (see, e.g., EP 388,151 A1, U.S. Pat. Nos. 5,624,821 and 5,648,260, all of which are hereby incorporated by reference in their entireties). Similar types of alterations may also be applied to murine immunoglobulins and immunoglobulins from other species. For example, it is possible to alter the affinity of an Fc region of an antibody (e.g., an IgG, such as a human IgG) for an FcR (e.g., Fc gamma R1) or for C1q binding by replacing the specified residue(s) with a residue(s) having an appropriate functionality on its side chain, or by introducing a charged functional group, such as glutamate or aspartate, or an aromatic nonpolar residue such as phenylalanine, tyrosine, tryptophan or alanine (see, e.g., U.S. Pat. No. 5,624,821).

Human antibodies to PBMC- and IBD-associated biomarkers of the invention may additionally be produced using transgenic nonhuman animals that are modified so as to produce fully human antibodies rather than the animal's endogenous antibodies in response to challenge by an antigen. See, e.g., PCT publication WO 94/02602. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. One embodiment of such a nonhuman animal is a mouse, and is termed the XENOMOUSE™ as disclosed in PCT publications WO 96/33735 and WO 96/34096. This animal produces B cells that secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

The binding capacity of an antibody of the invention may be measured by the following methods: Biacore analysis, enzyme linked immunosorbent assay (ELISA), X-ray crystallography, sequence analysis and scanning mutagenesis, and other methods that are known in the art.

Other protein-binding molecules may also be employed to modulate the activity of a PBMC- and IBD-associated biomarker. Such protein-binding molecules include small modular immunopharmaceutical (SMIP™) drugs (Trubion Pharmaceuticals, Seattle, Wash.). SMIPs are single-chain polypeptides composed of a binding domain for a cognate structure such as an antigen, a counterreceptor or the like, a hinge-region polypeptide having either one or no cysteine residues, and immunoglobulin CH2 and CH3 domains (see also www.trubion.com). SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Fragments of anti-biomarker antibodies may be produced by cleavage of the antibodies in accordance with methods well known in the art. For example, immunologically active F(ab′) and F(ab′)₂ fragments may be generated by treating the antibodies with an enzyme such as pepsin.

Anti-biomarker antibodies of the invention are also useful for isolating, purifying, and/or detecting PBMC- and IBD-associated biomarker polypeptides in the supernatant, cellular lysate, or on the cell surface. Antibodies disclosed in this invention can be used diagnostically to monitor levels of PBMC- and IBD-associated biomarker proteins as part of a clinical testing procedure, or to target a therapeutic modulator to a cell or tissue comprising the antigen of the anti-biomarker antibody. For example, a therapeutic of the invention, including but not limited to a small molecule, can be linked to the anti-biomarker antibody in order to target the therapeutic to a PBMC- and IBD-associated biomarker.

Detection of PBMC- and IBD-associated Biomarkers

The present invention provides methods for diagnosing, prognosing, and monitoring the progress of an IBD, e.g., Crohn's disease or ulcerative colitis, in a subject that directly or indirectly results from aberrant expression or activity levels of PBMC- and IBD-associated biomarkers by detecting such aberrant expression or activity levels of PBMC- and IBD-associated biomarkers, including, but not limited to, the use of such methods in human subjects. For example, these methods may be performed by utilizing prepackaged diagnostic kits comprising at least one of the group comprising PBMC- and IBD-associated biomarker polynucleotides and fragments thereof, PBMC- and IBD-associated biomarker polypeptides and derivatives thereof, antibodies to PBMC- and IBD-associated biomarkers, and modulators of PBMC- and IBD-associated biomarker polynucleotides and/or polypeptides as described herein, which may be conveniently used, for example, in a clinical setting. In addition, one of skill in the art would recognize that changes in expression or activity levels of one or more PBMC- and IBD-associated biomarkers may also be detected by well-known methods other than those described herein.

The diagnostic, prognostic, and monitoring assays of the present invention involve detecting and quantifying PBMC- and IBD-associated biomarker gene products in biological samples. PBMC- and IBD-associated biomarker gene products include, but are not limited to, PBMC- and IBD-associated biomarker mRNAs, cDNAs, and genomic DNAs, and PBMC- and IBD-associated biomarker polypeptides; such gene products can be measured using methods well known to those skilled in the art.

For example, MRNA of PBMC- and IBD-associated biomarkers can be directly detected and quantified using hybridization-based assays, such as Northern hybridization, in situ hybridization, dot and slot blots, and oligonucleotide arrays. Hybridization-based assays refer to assays in which a probe nucleic acid is hybridized to a target nucleic acid. In some formats, the target, the probe, or both are immobilized. The immobilized nucleic acid may be DNA, RNA, or other oligonucleotide or polynucleotides, and may comprise naturally or nonnaturally occurring nucleotides, nucleotide analogs, or backbones. Methods of selecting nucleic acid probe sequences for use in the present invention are based on the nucleic acid sequences of the PBMC- and IBD-associated biomarkers and are well known in the art.

Alternatively, mRNA of PBMC- and IBD-associated biomarkers may be amplified before detection and quantitation. Such amplification-based assays are well known in the art and include polymerase chain reaction (PCR), reverse-transcription-PCR (RT-PCR), PCR-enzyme-linked immunosorbent assay (PCR-ELISA), ligase chain reaction (LCR), self-sustained sequence replication, transcriptional amplification system, Q-beta Replicase, or any other polynucleotide amplification method. Primers and probes for producing and detecting amplified PBMC- and IBD-associated biomarker gene products may be readily designed and produced without undue experimentation by those of skill in the art based on the nucleic acid sequences of the PBMC- and IBD-associated biomarkers listed in Tables 1-5. Amplified PBMC- and IBD-associated gene products may be directly analyzed, for example, by gel electrophoresis; by hybridization to a probe nucleic acid; by sequencing; by detection of a fluorescent, phosphorescent, or radioactive signal; or by any of a variety of well-known methods. In addition, methods are known to those of skill in the art for increasing the signal produced by amplification of target nucleic acid sequences. One of skill in the art will recognize that whichever amplification method is used, a variety of quantitative methods known in the art (e.g., quantitative PCR (Q-PCR); also referred to herein as “real time PCR”, “quantitative real time PCR,” “quantitative real time reverse transcriptase polymerase chain reaction,” “quantitative real time RT-PCR,” and the like)) may be used if quantitation of PBMC- and IBD-associated gene products is desired.

PBMC- and IBD-associated biomarker polypeptides of the invention (or fragments thereof) can be detected using various well-known immunological assays employing anti-biomarker antibodies described above. Immunological assays refer to assays that utilize an antibody (e.g., polyclonal, monoclonal, chimeric, humanized, scFv, and fragments thereof) that specifically binds to a PBMC- and IBD-associated polypeptide (or fragment thereof). Such well-known immunological assays suitable for the practice of the present invention include ELISA, radioimmunoassay (RIA), immunoprecipitation, immunofluorescence, fluorescence-activated cell sorting (FACS) and Western blotting. In addition, an anti-biomarker antibody can be labeled with a radioactive biomarker whose presence and location in a subject can be detected by standard imaging techniques.

Each PBMC- and IBD-associated biomarker may be considered individually, although it is within the scope of the invention to provide combinations of two or more PBMC- and IBD-associated biomarkers for use in the methods and compositions of the invention to increase the confidence of the analysis. In one embodiment, the invention provides panels, e.g., models, of the PBMC- and IBD-associated biomarkers of the invention. A panel may comprise and/or consist essentially of 2-5, 5-15, 15-35, 35-50, 50-100, or more than 100 PBMC- and IBD-associated biomarkers. In one embodiment, a panel of the invention comprises and/or consists essentially of at least two PBMC- and IBD-associated biomarkers. In one embodiment, a panel of the invention comprises and/or consists essentially of at least three PBMC- and IBD-associated biomarkers. In one embodiment, a panel of the invention comprises and/or consists essentially of at least four PBMC- and IBD-associated biomarkers. In one embodiment, a panel of the invention comprises and/or consists essentially of at least five PBMC- and IBD-associated biomarkers. In one embodiment, a panel of the invention comprises and/or consists essentially of at least six PBMC- and IBD-associated biomarkers. In one embodiment, a panel of the invention comprises and/or consists essentially of at least seven PBMC- and IBD-associated biomarkers. In one embodiment, a panel of the invention comprises and/or consists essentially of at least eight PBMC- and IBD-associated biomarkers. In one embodiment, a panel of the invention comprises and/or consists essentially of at least nine PBMC- and IBD-associated biomarkers. In one embodiment, a panel of the invention comprises and/or consists essentially of at least ten PBMC- and IBD-associated biomarkers. In one embodiment, a panel of the invention comprises and/or consists essentially of at least eleven PBMC- and IBD-associated biomarkers. In one embodiment, a panel of the invention comprises and/or consists essentially of at least twelve PBMC- and IBD-associated biomarkers.

In another embodiment, panels of PBMC- and IBD-associated biomarkers are selected such that the biomarkers within any one panel share certain features. For example, the biomarkers of a first panel may each exhibit at least a 1.5-fold increase in quantity or activity in PBMCs from patients with inflammatory bowel disease, i.e., Crohn's disease or ulcerative colitis, as compared to PBMCs from a subject substantially free of IBD. Alternatively, biomarkers of a second panel may each exhibit differential regulation as compared to a first panel. Similarly, different panels of biomarkers may be composed of biomarkers from different functional categories (i.e., proteolysis, signal transduction, transcription, etc.) or samples (i.e., blood, kidney, spleen, lymph node, brain, intestine, colon, heart, urine, etc.), or may be selected to represent different stages of an inflammatory bowel disease, i.e., Crohn's disease or ulcerative colitis. In a preferred embodiment, panels of the invention comprise biomarkers from blood, and in particular, PBMCs. Panels of the PBMC- and IBD-associated biomarkers of the invention may be made by selecting as a panel the biomarkers categorized as Group I biomarkers, the biomarkers categorized as Group II biomarkers, the biomarkers categorized as Group III biomarkers, the biomarkers categorized as Group IV biomarkers, and/or the biomarkers categorized as Group V biomarkers. Panels may also be made by independently selecting biomarkers from Group I, Group II, Group III, Group IV, and/or Group V categorized biomarkers. In a preferred embodiment, a panel comprises and/or consists essentially of the set of PBMC- and IBD-associated biomarkers categorized as Group V biomarkers. A skilled artisan will also recognize that a panel of the invention may comprise and/or consist essentially of any number and any combination of PBMC- and IBD-associated biomarkers of the invention, particularly the Group V biomarkers of the invention. For example, a nonlimiting panel of the invention may comprise and/or consist essentially of the immunoglobulin heavy constant gamma 1 and immunoglobulin kappa constant PBMC- and IBD-associated biomarkers. Another nonlimiting panel of the invention may comprise and/or consist essentially of the human 28S ribosomal RNA 5′region, protein tyrosine phosphatase receptor type C-associated protein, H3 histone family member K, integrin beta 3 (platelet glycoprotein IIIa, antigen CD61) and H2B histone family member Q PBMC- and IBD-associated biomarkers. Another nonlimiting panel of the invention may comprise and/or consist essentially of the immunoglobulin heavy constant gamma 1, granzyme K, mutL homolog 3, lipocalin 2, CXCL5, serum deprivation response phosphatidylserine binding protein, and H3 histone family member K PBMC- and IBD-associated biomarkers. In one embodiment, a panel of the invention provides at least 70% accuracy (more preferably, at least 80% accuracy, most preferably at least 90% accuracy) in determining whether a patient has (1) IBD in the form of either Crohn's disease or ulcerative colitis, (2) Crohn's disease, and/or (3) ulcerative colitis, and/or in distinguishing between whether a patient with IBD has Crohn's disease or ulcerative colitis.

In addition to providing panels of PBMC- and IBD-associated biomarkers, it is within the scope of the invention to provide a panel of PBMC- and IBD-associated biomarkers conveniently coupled to a solid support. For example, PBMC- and IBD-associated biomarker polynucleotides of the invention may be coupled to an array (e.g., a biochip for hybridization analysis), to a resin (e.g., a resin that can be packed into a column for column chromatography), or a matrix (e.g., a nitrocellulose matrix for Northern blot analysis) using well-known methods in the art. Methods of making and using such arrays, including the use of such arrays with computer readable media (comprising PBMC- and IBD-associated biomarkers of the invention) and/or databases, e.g., a relational database, are well known in the art.

By providing such support, discrete analysis of the presence or activity in a sample of each PBMC- and IBD-associated biomarker selected for the panel may be detected. For example, in an array, polynucleotides complementary to each member of a panel of PBMC- and IBD-associated biomarkers may be individually attached to different known locations on the array using methods well known in the art. The array may be hybridized with, for example, polynucleotides extracted from a blood sample (preferably a PBMC sample) from a subject. The hybridization of polynucleotides from the sample with the array at any location on the array can be detected, and thus the presence or quantity of the PBMC- and IBD-associated biomarker(s) in the sample can be ascertained. Thus, not only tissue specificity, but also the level of expression of a panel of IBD biomarkers in the tissue is ascertainable. In a preferred embodiment, an array based on a biochip is employed. Similarly, ELISA analyses may be performed on immobilized antibodies specific for different polypeptide biomarkers hybridized to a protein sample from a subject.

In another embodiment, a reporter nucleic acid is utilized to detect the expression of one or more PBMC- and IBD-associated biomarkers of the invention. Such a reporter nucleic acid can be useful for high-throughput screens for agents that alter the expression profiles of peripheral blood mononuclear cells. The construction and use of such reporter assays are well known.

For example, the construction of a reporter for transcriptional regulation of a PBMC- and IBD-associated biomarker of the invention generally requires a regulatory sequence of PBMC- and IBD-associated biomarker, typically the promoter. The promoter can be obtained by a variety of routine methods. For example, a genomic library can be hybridized with a labeled probe consisting of the coding region of the nucleic acid to identify genomic library clones containing promoter sequences. The isolated clones can be sequenced to identify sequences upstream from the coding region. Another method is an amplification reaction using a primer that anneals to the 5′ end of the coding region of the PBMC- and IBD-associated biomarker polynucleotide. The amplification template can be, for example, restricted genomic nucleic acids to which anchor bubble adaptors have been ligated.

To construct the reporter, the promoter of the selected PBMC- and IBD-associated biomarker can be operably linked to the reporter nucleic acid, e.g., without utilizing the reading frame of the selected PBMC- and IBD-associated biomarker polynucleotide. The nucleic acid construct is transformed into tissue culture cells, e.g., peripheral blood mononuclear cells, by a transfection protocol to generate reporter cells.

Many of the well-known reporter nucleic acids may be used. In one embodiment, the reporter nucleic acid is green fluorescent protein. In a second embodiment, the reporter is β-galactosidase. In other embodiments, the reporter nucleic acid is alkaline phosphatase, β-lactamase, luciferase, chloramphenicol acetyltransferase, or other reporter nucleic acids known in the art. The reporter nucleic acid construct may be maintained on an episome or inserted into a chromosome by, for example, using targeted homologous recombination. Methods of making and using such reporter nucleic acids are well known.

Analysis with Group I-V Biomarkers

One of skill in the art will recognize that although the PBMC- and IBD-associated biomarkers of the invention may be categorized into five different groups, each individual biomarker is a PBMC- and IBD-associated biomarker of the invention. Additionally, a skilled artisan will recognize that the biomarkers are categorized into such groups for characterization purposes only. For example, the PBMC- and IBD-associated biomarkers of Group I have been determined to be biomarkers differentially expressed in PBMCs of patients with CD in common with PBMCs of patients with UC. Thus, these common biomarkers are categorized together to convey that they conveniently may be used together in assays of screening test compounds for treating an IBD, or may be used together for diagnosing, prognosing, and/or monitoring an IBD, without regard to whether the IBD is in the form of CD or UC. A skilled artisan will also recognize that the PBMC- and IBD-associated biomarkers categorized as Group II, Group III, Group IV, and/or Group V biomarkers may also be used to screen test compounds for, diagnose, prognose, and/or monitor an IBD, without regard to whether the IBD is in the form of CD or UC. However, it will be noted that Group II biomarkers, i.e., biomarkers included in the set of CD biomarkers, may be the optimal set to use in methods of screening test compounds for, diagnosing, prognosing, and/or monitoring an IBD when the IBD is in the form of CD. Conversely, Group III biomarkers, i.e., biomarkers included in the set of UC biomarkers, may be the optimal set to use in methods of screening test compounds for, diagnosing, prognosing, and/or monitoring an IBD when the IBD is in the form of UC. Also, Group IV biomarkers, i.e., biomarkers included in the set of CDvUC biomarkers, and particularly Group V biomarkers, i.e., biomarkers included in the set of classifying biomarkers, may be the optimal set(s) to use in methods of screening test compounds for, diagnosing, prognosing, and/or monitoring an IBD, when it is important to distinguish patients with CD from patients with UC.

Screening

In addition to methods of diagnosing, prognosing, and monitoring the progression of an IBD, the PBMC- and IBD-associated biomarker polynucleotides and polypeptides of the present invention may be used in screening assays to identify pharmacological agents, or lead compounds for agents, capable of regulating the activity of PBMC- and IBD-associated biomarkers, and thus, potentially capable of inhibiting or alleviating the symptoms of an IBD, i.e., Crohn's disease or ulcerative colitis. Such screening assays, including high throughput methods of screening, are well known in the art. For example, samples from subjects diagnosed with or suspected of having IBD, or samples containing PBMC- and IBD-associated biomarkers (either natural or recombinant) can be contacted with one of a plurality of test compounds (e.g., small organic molecules, biological agents), and the expression or activity levels of PBMC- and IBD-associated biomarkers in each of the treated samples can be compared to the expression or activity levels of PBMC- and IBD-associated biomarkers in untreated samples or in samples contacted with different test compounds to determine whether any of the test compounds provides: 1) a substantially decreased level of expression or activity of at least one PBMC- and IBD-associated biomarker, thereby indicating an inhibitor of the activity of at least one PBMC- and IBD-associated biomarker, or 2) a substantially increased level of expression or activity of at least one PBMC- and IBD-associated biomarker, thereby indicating an agent that increases the activity of at least one PBMC- and IBD-associated biomarker. In a preferred embodiment, the identification of test compounds capable of regulating the activity of at least one PBMC- and IBD-associated biomarker is performed using high-throughput screening assays, such as provided by BIACORE®(Biacore International AB, Uppsala, Sweden), or BRET (bioluminescence resonance energy transfer), and FRET (fluorescence resonance energy transfer) assays, as well as ELISA and cell-based assays.

In addition, the invention is further directed to a method of screening for test compounds capable of regulating the binding of a PBMC- and IBD-associated biomarker to a binding partner, the method carried out by combining the test compound, the PBMC- and IBD-associated protein, and the binding partner and determining whether binding of the binding partner and PBMC- and IBD-associated protein occurs, and how such binding is positively or negatively modulated by the test compound.

As mentioned above, the agent capable of regulating the activity of a PBMC- and IBD-associated biomarker may be any of a variety of naturally occurring or synthetic compounds, biomolecules, proteins, peptides, oligopeptides, polysaccharides, nucleotides or polynucleotides. The test compound may be, e.g., a small molecule or a biological agent. As discussed below, test compounds may be provided from a variety of libraries well known in the art.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the finctionalities of peptides, but with novel, nonpeptide backbones that are resistant to enzymatic degradation yet remain bioactive; see, e.g., Zuckermann et al. (1994) J Med. Chem. 37:2678-85); spatially addressable parallel solid phase or solution phase libraries; synthetic library methods requiring deconvolution; the “one-bead, one-compound” library method; and synthetic library methods using affinity chromatography selection. The biological library and peptoid library approaches are limited to peptide libraries, while the other four approaches are applicable to peptide, nonpeptide oligomer or small molecule libraries of compounds (see generally, e.g., Lam (1997) Anticancer Drug Des. 12(3):145-67).

Methods for Diagnosing, Prognosing and Monitoring the Progress of an Inflammatory Bowel Disease

“Diagnostic” or “diagnosing” means identifying the presence or absence of a pathologic condition. Diagnostic methods involve detecting substantially modulated (i.e., aberrant) expression of PBMC- and IBD-associated biomarkers by determining a test amount of PBMC- and IBD-associated biomarker gene products (e.g., mRNA, cDNA, or polypeptide, including fragments thereof) in a biological sample from a subject (human or nonhuman mammal), and comparing the test amount with the normal amount or range (i.e., an amount or range from an individual(s) known not to suffer from IBD) for the PBMC- and IBD-associated biomarker gene product.

In one embodiment, the levels of PBMC- and IBD-associated biomarkers in the two samples are compared, and aberrant expression of one or more PBMC- and IBD-associated biomarkers in the test sample indicates IBD. In other embodiments, the aberrant expression of 2, 3, 4 or more biomarkers indicates a severe case of IBD. In another embodiment, the aberrant expression one or more biomarkers indicates a likelihood of IBD, and aberrant expression of 2, 3, 4 or more biomarkers indicates an increased likelihood of IBD. In another aspect, the invention provides biomarkers whose quantity or activity is correlated with different manifestations or severity or types of IBD. For example, aberrant expression of the PBMC- and IBD-associated biomarkers in Table 5, as indicated, may correlate with a diagnosis of Crohn's disease or ulcerative colitis. The subsequent level of expression may further be compared to different expression profiles of various stages of the disorder to confirm whether the subject has a matching profile. Although a particular diagnostic method may not provide a definitive diagnosis of IBD, it suffices if the method provides a positive indication that aids in diagnosis.

The present invention also provides methods for prognosing IBD by detecting aberrant expression or activity levels of at least one PBMC- and IBD-associated biomarker. “Prognostic” or “prognosing” means predicting the probable development and/or severity of a pathologic condition. Prognostic methods involve determining the test amount of at least one PBMC- and IBD-associated biomarker gene product in a biological sample from a subject, and comparing the test amount to a prognostic amount or range (i.e., an amount or range from individuals with varying severities of IBD) for the PBMC- and IBD-associated biomarker gene product. Various amounts of the PBMC- and IBD-associated biomarker gene product in a test sample are consistent with certain prognoses for IBD, Crohn's disease, and/or ulcerative colitis. The detection of an amount of PBMC- and IBD-associated biomarker gene product at a particular prognostic level provides for a prognosis for the subject. In one embodiment of the present invention, as related to IBD (or a particular form of IBD), substantially upregulated expression or activity of one or more PBMC- and IBD-associated biomarkers is typically correlated with an abnormal increase. In another embodiment of the present invention, as related to IBD (or a particular form of IBD), substantially downregulated expression or activity of one or more PBMC- and IBD-associated biomarkers is typically correlated with an abnormal decrease.

In addition, the prognostic assays described herein can be used to determine whether a subject can be administered an agent (e.g., an agonist, antagonist, peptidomimetic, protein, peptide, polynucleotide, small molecule, or other drug candidate) to treat or prevent IBD associated with aberrant PBMC- and IBD-associated biomarker expression or activity. Accordingly, regulation of a PBMC- and IBD-associated biomarker, such as PAI-2, to normal levels (e.g., levels similar or substantially similar to tissue substantially free of IBD) may allow for amelioration of IBD.

In relation to the field of gastroenterology, prognostic assays can be devised to determine whether a subject undergoing treatment for such disorder has a poor outlook for long-term survival or disease progression. In a preferred embodiment, prognosis can be determined shortly after diagnosis, i.e., within a few days. By establishing expression profiles of different stages of IBD, or of a particular form of IBD (e.g., Crohn's disease or ulcerative colitis), from onset to acute disease, an expression pattern may emerge to correlate a particular expression profile to increased likelihood of a poor prognosis. The prognosis may then be used to devise a more aggressive treatment program to avert chronic IBD and enhance the likelihood of long-term survival and well-being.

In a preferred embodiment of the invention, the disclosed molecules and methods are used on a biological sample to detect, in PBMC- and IBD-associated biomarker genes, the presence of one or more genetic alterations well known to result in aberrant expression of PBMC- and IBD-associated biomarkers. Such detecting can be used to determine the severity of IBD or to prognosticate the potential for IBD due to modulated expression or activity of PBMC- and IBD-associated biomarkers. In a further specific embodiment, one or more genetic alterations are correlated with the prognosis or susceptibility of a subject to IBD. Genetic alterations in a PBMC- and IBD-associated biomarker gene from a sample can be identified by well-known methods in the art, including, but not limited to, sequencing reactions, electrophoretic mobility assays, and oligonucleotide hybridizations.

The present invention also provides methods for monitoring the progress or course of IBD, Crohn's disease, and/or ulcerative colitis, by monitoring the expression or activity of PBMC- and IBD-associated biomarkers. Monitoring methods involve determining the test amount of a PBMC- and IBD-associated biomarker gene product in biological samples taken from a subject at a first and second time, and comparing the amounts. A change in the amount of a PBMC- and IBD-associated biomarker, or changes in the amounts of PBMC- and IBD-associated biomarkers, between the first and second time indicates a change in the course of the IBD. Such monitoring assays are also useful for evaluating the efficacy of a particular therapeutic intervention in patients (e.g., during clinical trials), i.e., evaluating the modulation of PBMC- and IBD-associated biomarkers in response to therapeutic agents provided herein.

It will be appreciated that the assay methods of the present invention do not necessarily require measurement of absolute values of PBMC- and IBD-associated biomarker gene products because relative values are sufficient for many applications of these methods. It will also be appreciated that in addition to the quantity or abundance of PBMC- and IBD-associated biomarker gene products, variant or abnormal PBMC- and IBD-associated biomarker gene products or their expression patterns (e.g., mutated transcripts, truncated polypeptides) may be identified by comparison to normal gene products and expression patterns.

Methods of Treatment

The present invention provides for both prophylactic and therapeutic methods of treating a subject at risk for, susceptible to, or diagnosed with IBD, Crohn's disease, and/or ulcerative colitis. Subjects at risk, susceptible to, or diagnosed with an IBD that is caused or contributed to by aberrant PBMC- and IBD-associated biomarker expression or activity can be identified by, for example, any of the diagnostic or prognostic assays as described herein, or a combination thereof. In one aspect, the invention provides prophylactic methods for preventing, in a subject, IBD associated with aberrant PBMC- and IBD-associated biomarker expression or activity, by administering to the subject a PBMC- and IBD-associated biomarker protein or an agent, which regulates PBMC- and IBD-associated biomarker protein expression or activity. Administration of a prophylactic agent can occur prior to the manifestation of symptoms characteristic of the differential PBMC- and IBD-associated biomarker protein expression, such that IBD is prevented or, alternatively, delayed in its progression. Another aspect of the invention pertains to therapeutic methods of regulating expression or activity levels of PBMC- and IBD-associated biomarkers for therapeutic purposes. Accordingly, in an exemplary embodiment, this regulatory method of the invention involves contacting cells (e.g., PBMCs) with an agent that regulates the expression level(s) or one or more of the activities of PBMC- and IBD-associated biomarkers.

An agent that regulates expression or activity levels of PBMC- and IBD-associated biomarkers, i.e., a regulatory agent of at least one PBMC- and IBD-associated biomarker, may be an agent as described herein, such as a PBMC- and IBD-associated biomarker polynucleotide (including related PBMC- and IBD-associated biomarker polynucleotides (e.g., inhibitory polynucleotides)), a PBMC- and IBD-associated biomarker protein, a naturally occurring target molecule of a PBMC- and IBD-associated biomarker protein (e.g., a PBMC- and IBD-associated biomarker protein substrate), an anti-biomarker antibody, a PBMC-and IBD-associated biomarker agonist, a PBMC-and IBD-associated biomarker antagonist, or other small molecule. The appropriate agent can be determined based on screening assays described herein.

These regulatory methods can be performed in vitro (e.g., by culturing PBMCs with the agent) or, alternatively, in vivo (e.g., by administering the regulatory agent to a subject). In one embodiment, the method involves administering a PBMC- and IBD-associated biomarker protein or polynucleotide molecule or a PBMC- and IBD-associated agonist as therapy to compensate for substantially reduced or aberrant PBMC- and IBD-associated biomarker protein expression or activity. Stimulation or upregulation of PBMC- and IBD-associated biomarker activity is desirable in situations in which PBMC- and IBD-associated biomarker protein is substantially downregulated and/or in which increased PBMC- and IBD-associated biomarker activity is likely to have a beneficial effect.

In another embodiment, the method involves the administration of an inhibitory polynucleotide or polypeptide as therapy to compensate for substantially increased or aberrant PBMC- and IBD-associated biomarker expression or activity. Inhibition or downregulation of PBMC- and IBD-associated biomarker activity is desirable in situations in which PBMC- and IBD-associated biomarker expression or activity is substantially upregulated and/or in which decreased PBMC- and IBD-associated biomarker activity is likely to have a beneficial effect.

Several pharmacogenomic approaches to be considered in determining whether to administer a regulatory agent of at least one PBMC- and IBD-associated biomarker are well known to one of skill in the art and include genome-wide association, candidate gene approach, and gene expression profiling. A pharmaceutical composition of the invention is formulated to be compatible with its intended route of administration (e.g., oral compositions generally include an inert diluent or an edible carrier). Other nonlimiting examples of routes of administration include parenteral (e.g., intravenous, subcutaneous, intramuscular), oral (e.g., inhalation), rectal, transdermal (topical), and transmucosal administration. The pharmaceutical compositions compatible with each intended route are well known in the art.

A regulatory agent of at least one PBMC- and IBD-associated biomarker may be used as a pharmaceutical composition when combined with a pharmaceutically acceptable carrier(s). Such a composition may contain, in addition to the regulatory agent of at least one PBMC- and IBD-associated biomarker and a carrier(s), various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration.

The pharmaceutical composition of the invention may also contain cytokines, lymphokines, or other hematopoietic factors such as M-CSF, GM-CSF, IL-1, IL-2, IL-3, IL-4, IL-5, IL-6, IL-7, IL-8, IL-9, IL-10, IL-11, IL-12, IL14, IL-15, G-CSF, stem cell factor, and erythropoietin. The pharmaceutical composition may also include anticytokine antibodies, thrombolytic or antithrombotic factors such as plasminogen activator and Factor VIII, and/or other anti-inflammatory agents. Such additional factors and/or agents may be included in the pharmaceutical composition to produce a synergistic effect with a regulatory agent of at least one PBMC- and IBD-associated biomarker, or to minimize side effects caused by the regulatory agent. In addition, a composition of the invention may also include (in addition to a regulatory agent of at least one PBMC- and IBD-associated biomarker of the invention) known agent(s) used to treat IBD, e.g., sulfasalazine, 5-ASA, steroids, etc. Conversely, a regulatory agent to at least one PBMC- and IBD-associated biomarker may be included in formulations of the particular cytokine, lymphokine, other hematopoietic factor, thrombolytic or antithrombotic factor, or anti-inflammatory agent to minimize side effects of the cytokine, lymphokine, other hematopoietic factor, thrombolytic or antithrombotic factor, or anti-inflammatory agent.

The pharmaceutical composition of the invention may be in the form of a liposome in which a regulatory agent of at least one PBMC- and IBD-associated biomarker is combined, in addition to other pharmaceutically acceptable carriers, with amphipathic agents such as lipids that exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, etc. Preparation of such liposomal formulations is well known by those of skill in the art.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of conditions related to IBD, etc. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of a regulatory agent of at least one PBMC- and IBD-associated biomarker is administered to a subject, e.g., a mammal (e.g., a human). A regulatory agent may be administered in accordance with the method of the invention either alone or in combination with other therapies, such as treatments employing cytokines, lymphokines or other hematopoietic factors, or anti-inflammatory agents. When coadministered with one or more agents, a regulatory agent of at least one PBMC- and IBD-associated biomarker may be administered either simultaneously with the other agent(s), or sequentially. If administered sequentially, the attending physician will decide on the appropriate sequence of administering, e.g., a regulatory agent of at least one PBMC- and IBD-associated biomarker, in combination with other regulatory agents of at least one PBMC- and IBD-associated biomarker, or in combination with other agents.

In one embodiment, the regulatory agent(s) of at least one PBMC- and IBD-associated biomarker of the invention, e.g., pharmaceutical compositions thereof, are administered in combination therapy, i.e., combined with other agents, e.g., therapeutic agents, that are useful for treating pathological conditions or disorders, such as immune and/or inflammatory disorders. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment or in the subject.

Combination Therapy

Combination therapy can include, e.g., a regulatory agent of a PBMC- and IBD-associated biomarker coformulated with, and/or coadministered with, at least one additional therapeutic agent. Additional agents may include at least one cytokine inhibitor, growth factor inhibitor, immunosuppressant, anti-inflammatory agent, metabolic inhibitor, enzyme inhibitor, cytotoxic agent, or cytostatic agent, as described in more detail below. Such combination therapies may advantageously utilize lower dosages of the administered therapeutic agents, thus avoiding possible toxicities or complications associated with the various monotherapies. Moreover, the therapeutic agents disclosed herein act on pathways that differ from the IBD injury pathway, and thus are expected to enhance and/or synergize with the effects of the at least one regulatory agent of a PBMC- and IBD-associated biomarker.

Therapeutic agents used in combination with a regulatory agent of a PBMC- and IBD-associated biomarker may be those agents that interfere at different stages in an inflammatory response. In one embodiment, at least one regulatory agent of a PBMC- and IBD-associated biomarker described herein may be coformulated with, and/or coadministered with, at least one cytokine and/or growth factor antagonist. The cytokine and/or growth factor antagonists may include soluble receptors, peptide inhibitors, small molecules, ligand fusions, antibodies (that bind cytokines or growth factors or their receptors or other cell surface molecules), and “anti-inflammatory cytokines” and agonists thereof.

Nonlimiting examples of the agents that can be used in combination with the regulatory agents of PBMC- and IBD-associated biomarkers described herein, include, but are not limited to, antagonists of at least one interleukin (e.g., IL-1, IL-2, IL-6, IL-7, IL-8, IL-12, IL-13, IL-15, IL-16, IL-17, IL-18, IL-21, and IL-22); cytokine (e.g., TNFα, LT, EMAP-II, and GM-CSF); or growth factor (e.g., FGF and PDGF). The agents may also include, but are not limited to, antagonists of at least one receptor for an interleukin, cytokine, and growth factor. Regulatory agents of PBMC- and IBD-associated biomarkers can also be combined with inhibitors of, e.g., antibodies to, cell surface molecules such as CD2, CD3, CD4, CD8, CD20 (e.g., the CD20 inhibitor rituximab (RITUXAN®)), CD25, CD28, CD30, CD40, CD45, CD69, CD80 (B7.1), CD86 (B7.2), CD90, or their ligands, including CD154 (gp39 or CD40L), or LFA-1/ICAM-1 and VLA-4/VCAM-1 (Yusuf-Makagiansar et al. (2002) Med. Res. Rev. 22:146-67). Other compounds that can be used in combination with regulatory agents of PBMC- and IBD-associated biomarkers described herein may include antagonists of the receptors for IL-1, IL-12, TNFα, IL-15, IL-17, IL-18, IL-21 and IL-22.

Examples of agents useful in combination therapies with a regulatory agent of a PBMC- and IBD-associated biomarker include IL-12 antagonists (such as antibodies that bind IL-12 (see e.g., WO 00/56772)); IL-12 receptor inhibitors (such as antibodies to the IL-12 receptor); and soluble IL-12 receptor and fragments thereof. Examples of IL-15 antagonists include antibodies against IL-15 or its receptor, soluble fragments of the IL-15 receptor, and IL- 15-binding proteins. Examples of IL-18 antagonists include antibodies to IL-18, soluble fragments of the IL-18 receptor, and IL-18 binding proteins (IL-18BP, Mallat et al. (2001) Circ. Res. 89:E41-45). Examples of IL-1 antagonists include interleukin-1-converting enzyme (ICE) inhibitors (such as Vx740), IL-1 antagonists (e.g., IL-1RA (anakinra (KINERE™), Amgen)), sIL-1RII (Immunex), and anti-IL-1 receptor antibodies.

Examples of TNF antagonists include antibodies to TNF (e.g., human TNFα), such as D2E7 (human anti-TNFα antibody, U.S. Pat. No. 6,258,562, HUMIRA™, Abbott Labs); CDP-571/CDP-870/BAY-10-3356 (humanized anti-TNFα antibodies, Celltech/Pharmacia); cA2 (chimeric anti-TNFα antibody, REMICADE™, Centocor); and anti-TNF antibody fragments (e.g., CPD870). Other examples include soluble TNF receptor (e.g., human p55 or p75) fragments and derivatives, such as p55 kD TNFR-IgG (55 kD TNF receptor-IgG fusion protein, LENERCEPT™) and 75 kd TNFR-IgG (75 kD TNF receptor-IgG fusion protein, ENBREL™ (etanercept-Immunex)). See, e.g., van der Poll et al. (1997) Blood 89:3727-34; Mori et al. (1996) J. Immunol. 157:3178-82. Further examples include enzyme antagonists (e.g., TNFα converting enzyme inhibitors (TACE) such as alpha-sulfonyl hydroxamic acid derivative (WO 01/55112) or N-hydroxyformamide inhibitors (GW 3333, -005, or -022, GlaxoSmithKline) and TNF-bp/s-TNFR (soluble TNF binding protein, see, e.g., Lantz et al. (1991) J. Clin. Invest. 88:2026-31; Kapadia et al. (1995) Amer. J. Physiol. Heart Circ. Phys. 268:H517-25). TNF antagonists may be soluble TNF receptor (e.g., human p55 or p75) fragments and derivatives, such as 75 kd TNFR-IgG; and TNFα converting enzyme (TACE) inhibitors.

In other embodiments, the regulatory agents of PBMC- and IBD-associated biomarkers described herein can be administered in combination with at least one of the following: IL-13 antagonists, such as soluble IL-13 receptors and/or anti-IL-13 antibodies; and IL-2 antagonists, such as IL-2 fusion proteins (e.g., DAB 486-IL-2 and/or DAB 389-IL-2 made by Seragen, see, e.g., Sewell et al. (1993) Arthritis Rheum. 36:1223-33) and anti-IL-2R antibodies (e.g., anti-Tac-H humanized antibody, Protein Design Labs, see Junghans et al. (1990) Cancer Res. 50:1495-502). Another combination includes regulatory agents of PBMC- and IBD-associated biomarkers in combination with nondepleting anti-CD4 inhibitors such as IDEC-CE9.1/SB 210396 (anti-CD4 antibody, GlaxoSmithKline). Yet other combinations include regulatory agents of PBMC- and IBD-associated biomarkers with CD80 (B7.1) and CD86 (B7.2) costimulatory pathway antagonists (such as antibodies, soluble receptors, or antagonistic ligands); P-selectin glycoprotein ligand (PSGL) and PSGL-1 inhibitors (such as antibodies to PSGL and/or PSGL-1 and small molecule inhibitors); T cell- and B cell-depleting agents (such as anti-CD4 or anti-CD22 antibodies), and anti-inflammatory cytokines and agonists thereof (e.g., antibodies). The anti-inflammatory cytokines may include IL-4 (e.g., Schering-Plough Biopharma); IL-10 (e.g., SCH 52000, recombinant IL-10, Schering-Plough Biopharma); IL-11; IL-13; and TGFβ or agonists thereof (e.g., agonist antibodies).

In other embodiments, at least one regulatory agent of a PBMC- and IBD-associated biomarker can be coformulated with, and/or coadministered with, at least one anti-inflammatory drug, immunosuppressant, metabolic inhibitor, and enzymatic inhibitor. Nonlimiting examples of the drugs or inhibitors that can be used in combination with the regulatory agents of PBMC- and IBD-associated biomarkers described herein include, but are not limited to, at least one of: nonsteroidal anti-inflammatory drugs (NSAIDs) (including, but not limited to, aspirin, salsalate, diflunisal, ibuprofen, ketoprofen, nabumetone, piroxicam, naproxen, diclofenac, indomethacin, sulindac, tolmetin, etodolac, ketorolac, oxaprozin, tenidap, meloxicam, piroxicam, aceclofenac, tolmetin, tiaprofenic acid, nimesulide, etc.); sulfasalazine; corticosteroids (such as prednisolone); cytokine suppressive anti-inflammatory drugs (CSAIDs); inhibitors of nucleotide biosynthesis (such as inhibitors of purine biosynthesis (e.g., folate antagonist such as methotrexate)); and inhibitors of pyrimidine biosynthesis, e.g., a dihydroorotate dehydrogenase (DHODH) inhibitor such as leflunomide (see, e.g., Kraan et al. (2004) Ann. Rheum. Dis. 63:1056-61). Therapeutic agents for use in combination with regulatory agents of at least one PBMC- and IBD-associated biomarker may include one or more NSAIDs, CSAIDs, DHODH inhibitors (such as leflunomide), and folate antagonists (such as methotrexate).

Examples of additional agents that may be used in combination with regulatory agents of PBMC- and IBD-associated biomarkers include at least one of: corticosteroid (oral, inhaled and local injection); immunosuppressant (such as cyclosporin and tacrolimus (FK-506)); an mTOR inhibitor (such as sirolimus (rapamycin) or a rapamycin analog and/or derivative, e.g., ester rapamycin derivative such as CCI-779 (see, e.g., Elit (2002) Curr. Opin. Investig. Drugs 3:1249-53; Huang et al. (2002) Curr. Opin. Investig. Drugs 3:295-304)); an agent which interferes with the signaling of proinflammatory cytokines such as TNFα and IL-1 (e.g., an IRAK, NIK, IKK, p38 or MAP kinase inhibitor); TPL-2, Mk-2 and NFκb inhibitors; COX-2 inhibitors (e.g., celecoxib, rofecoxib, etc., and variants thereof); phosphodiesterase inhibitors (such as Rolipram); phospholipase inhibitors (e.g., an inhibitor of cytosolic phospholipase 2 (cPLA2) such as trifluoromethyl ketone analogs (U.S. Pat. No. 6,350,892)); inhibitors of vascular endothelial cell growth factor (VEGF); inhibitors of the VEGF receptor; inhibitors of angiogenesis; RAGE and soluble RAGE; estrogen receptor beta (ERB) agonists, ERB-NFκb antagonists; interferon-β (for example, IFNβ-1a and IFNβ-1b); copaxone; and corticosteroids.

Other useful therapeutic agents that may be combined with one or more regulatory agent(s) of a PBMC- and IBD-associated biomarker include: budenoside; epidermal growth factor; aminosalicylates; 6-mercaptopurine; azathioprine; metronidazole; lipoxygenase inhibitors; mesalamine; olsalazine; balsalazide; antioxidants; thromboxane inhibitors; growth factors; elastase inhibitors; pyridinyl-imidazole compounds; glucuronide- or dextran-conjugated prodrugs of prednisolone; dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; antagonists of platelet activating factor (PAF); ciprofloxacin; lignocaine; cyclosporin A; hydroxychloroquine (PLAQUENIL™); minocycline (MINOCIN™); and anakinra (KINERET™).

Choosing a particular therapeutic agent for administration in combination with regulatory agents of PBMC- and IBD-associated biomarkers of the invention will largely depend on factors such as the particular subject, the desired target, and chosen length of treatment. Such decisions are well within the skill and knowledge of one skilled in the art.

Additional examples of therapeutic agents that can be combined with a regulatory agent of a PBMC- and IBD-associated biomarker include one or more of: 6-mercaptopurines (6-MP); azathioprine; sulfasalazine; mesalazine; olsalazine; chloroquine, hydroxychloroquine (PLAQUENIL®); penicillamine; aurothiornalate (intramuscular and oral); azathioprine; colchicine; beta-2 adrenoreceptor agonists (salbutamol, terbutaline, salmeterol); xanthines (theophylline, aminophylline); cromoglycate; nedocromil; ketotifen; ipratropium and oxitropium; mycophenolate mofetil; adenosine agonists; antithrombotic agents; complement inhibitors; and adrenergic agents.

In one embodiment, a regulatory agent of a PBMC- and IBD-associated biomarker can be used in combination with one or more antibodies directed at other targets involved in regulating immune responses. Nonlimiting examples of agents for treating or preventing immune responses with which a regulatory agent of a PBMC- and IBD-associated biomarker of the invention can be combined include the following: antibodies against other cell surface molecules, including but not limited to CD25 (interleukin-2 receptor-a), CD11a (LFA-1), CD54 (ICAM-1), CD4, CD45, CD28, CTLA4, ICOSL, ICOS, CD80 (B7.1), and/or CD86 (B7.2). In yet another embodiment, a regulatory agent of a PBMC- and IBD-associated biomarker is used in combination with one or more general immunosuppressive agents, such as cyclosporine A or FK506. In another embodiment, a regulatory agent of a PBMC- and IBD-associated biomarker is used in combination with a CTLA4 agonist, e.g., (e.g., CTLA4 Ig -abatacept (ORENCIA®)).

A pharmaceutical composition of the invention is formulated to be compatible with its intended route of administration. Methods to accomplish the administration are known to those of ordinary skill in the art. It may also be possible to obtain compositions which may be topically or orally administered, or which may be capable of transmission across mucous membranes. Administration of a modulator of the invention used in the pharmaceutical composition to practice the method of the present invention can be carried out in a variety of conventional ways, such as oral ingestion, inhalation, cutaneous, subcutaneous, intravenous injection, rectal enema, insertion of a suppository, etc.

Solutions or suspensions used for intradermal or subcutaneous application typically include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium. bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, CREMAPHORE™ EL (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride, in the composition. Prolonged absorption of the injectable compositions can be brought about by including in the composition an agent which delays absorption, for example, aluminum monostearate and gelatin.

When a therapeutically effective amount of a regulatory agent of at least one PBMC- and IBD-associated biomarker is administered orally, the binding agent will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% binding agent, and preferably from about 25 to 90% binding agent. When administered in liquid form, a liquid carrier such as water, petroleum, oils of animal or plant origin such as peanut oil (albeit keeping in mind the frequency of peanut allergies in the population), mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition contains from about 0.5 to 90% by weight of the binding agent, and preferably from about 1 to 50% by weight of the binding agent.

When a therapeutically effective amount of a regulatory agent of at least one PBMC- and IBD-associated biomarker is administered by intravenous, cutaneous or subcutaneous injection, the regulatory agent will be in the form of a pyrogen-free, parenterally acceptable aqueous solution. The preparation of such parenterally acceptable protein solutions, having due regard for pH, isotonicity, stability, and the like, is within the skill of those in the art. A preferred pharmaceutical composition for intravenous, cutaneous, or subcutaneous injection should contain, in addition to the regulatory agent of at least one PBMC- and IBD-associated biomarker, an isotonic vehicle such as sodium chloride injection, Ringer's injection, dextrose injection, dextrose and sodium chloride injection, lactated Ringer's injection, or other vehicle as known in the art. The pharmaceutical composition of the present invention may also contain stabilizers, preservatives, buffers, antioxidants, or other additive known to those of skill in the art.

The amount of a regulatory agent of at least one PBMC- and IBD-associated biomarker in the pharmaceutical composition of the present invention will depend upon the nature and severity of the condition being treated, and on the nature of prior treatments that the patient has undergone. Ultimately, the attending physician will decide the amount of the regulatory agent of at least one PBMC- and IBD-associated biomarker with which to treat each individual patient. Initially, the attending physician will administer low doses of the regulatory agent of at least one PBMC- and IBD-associated biomarker and observe the patient's response. Larger doses of the regulatory agent of at least one PBMC- and IBD-associated biomarker may be administered until the optimal therapeutic effect is obtained for the patient, and at that point the dosage is not generally increased further. It is contemplated that the various pharmaceutical compositions used to practice the method of the present invention should contain about 0.1 μg to about 100 mg per kg body weight.

The duration of intravenous (i.v.) therapy using a pharmaceutical composition of the present invention will vary, depending on the severity of the disease being treated and the condition and potential idiosyncratic response of each individual patient. It is contemplated that the duration of each application of the regulatory agent of at least one PBMC- and IBD-associated biomarker may be in the range of 12 to 24 hours of continuous i.v. administration, or some other appropriate period. Also contemplated is subcutaneous (s.c.), suppository, etc. therapy using a pharmaceutical composition of the present invention. These therapies can be administered daily, weekly, or, more preferably, biweekly, or monthly. It is also contemplated that where the regulatory agent of at least one PBMC- and IBD-associated biomarker is a small molecule, the therapies may be administered daily, twice a day, three times a day, etc. Ultimately the attending physician will decide on the appropriate duration of therapy, or therapy with a small molecule, and the timing of administration of the therapy, using the pharmaceutical composition of the present invention.

Kits

The invention also provides kits for determining the prognosis for long-term survival or well-being in a subject having an inflammatory bowel disease, the kit comprising reagents for assessing expression of the biomarkers of the invention. Kits for diagnosis and monitoring are also contemplated. Preferably, the reagents may comprise one or more anti-biomarker antibody or fragment thereof, wherein the antibody or fragment thereof specifically binds with a protein corresponding to a PBMC- and IBD-associated biomarker. Optionally, the kits may comprise a polynucleotide probe wherein the probe specifically binds with a transcribed polynucleotide corresponding to a PBMC- and IBD-associated biomarker listed in Tables 1-5. The kits may also include a panel of PBMC- and IBD-associated biomarkers, which may be arranged as an array on a biochip, such as, for example, a GENECHIP®.

The invention further provides kits for assessing the suitability of each of a plurality of compounds for inhibiting an inflammatory bowel disease in a subject. Such kits include a plurality of compounds to be tested, and a reagent (i.e., an antibody specific to corresponding proteins, or a probe or primer specific to corresponding polynucleotides) for assessing expression of a PBMC- and IBD-associated biomarker listed in Tables 1-5.

Modifications to the above-described compositions and methods of the invention, according to standard techniques, will be readily apparent to one skilled in the art and are meant to be encompassed by the invention.

This invention is further illustrated by the following examples, which should not be construed as limiting. The contents of all references, patents and patent applications cited throughout this application are hereby incorporated by reference herein.

### EXAMPLES

The Examples which follow are set forth to aid in the understanding of the invention but are not intended to, and should not be construed to, limit its scope in any way. The Examples do not include detailed descriptions of conventional methods, such as isolation of peripheral blood mononuclear cells from healthy volunteers and patients afflicted with inflammatory bowel disease. Such methods are well known to those of ordinary skill in the art.

### Example 1

### Materials and Methods

### Example 1.1

Patient Information and Clinical Assessments

Blood samples for pharmacogenomic analysis were collected at North American and European clinical sites from a total of 42 apparently healthy individuals, 59 CD patients and 26 UC patients. Each clinical site's Institutional Review Board or Ethics Committee approved this study, and no procedures were performed prior to obtaining informed consent from each patient.

A comparison of the demographic characteristics of individuals in the present study is presented in Table 7.

TABLE 7
Demographic characteristics of normal disease-free individuals (C) and
subjects with IBD, in the form of Crohn's disease (CD) or ulcerative
colitis (UC)

| | | | | | CD |
|---|---|---|---|---|---|
| | | | | C v IBD | v UC |
| Type | CD | UC | C | p-value | p-value |
| Number | 59 | 26 | 42 | | |
| of | | | | | |
| Samples | | | | | |
| Age | 41.3 | 46.7 | 44.1 | 0.96¹ | 0.055¹ |
| (mean) | | | | | |
| Sex | 21 Males | 8 Males | 24 Males | 0.014² | 0.66² |
| | 38 Females | 18 Females | 18 Females | | |
| Race | 51 Caucasian | 22 Caucasian | 40 Caucasian | 0.09³ | 0.82³ |
| | 7 Black | 3 Black | 1 Asian | | |
| | 1 Hispanic | 1 Hispanic | 1 Indian | | |

¹p-value calculated using two-sided t-test with t-statistic based on ANOVA error estimate.
²p-value calculated using Likelihood Ratio chi-square test comparing male to female frequencies among groups.
³p-value calculated using Likelihood Ratio chi-square test comparing Caucasian to non- Caucasian frequencies among groups.

Healthy subjects (24 males, 18 females) were predominantly Caucasian and ranged in age from 25 to 60 years. CD patients (21 males, 38 females) were predominantly Caucasian and ranged in age from 20 to 65 years, with Crohn's disease activity index scores (CDAI) ranging between 220 and 400, and with an abdominal pain rating of ≧25 and/or a diarrhea rating of ≧25. Diagnosis of CD for at least 6 months was confirmed by radiological studies, endoscopy with histological examination, or surgical pathology; patients with a diagnosis of Crohn's disease were included if the diagnosis was confirmed by a biopsy. UC patients (8 males, 18 females) were predominantly Caucasian and ranged in age from 25 to 73 years, and had scores from the Physician's Global Assessment of the Mayo Ulcerative Colitis Scoring System (MUCSS) ranging from mild to moderate (scores of 1 or 2). The diagnosis of left-sided UC was provided by endoscopy with biopsy, in addition to standard clinical criteria.

Proportions of females to males were significantly different between the healthy and IBD populations, but not distinct between the two IBD populations; neither race (Caucasian vs. non-Caucasian) nor age differed significantly between healthy and IBD populations, or between the two IBD populations (all at the p<0.05 level). Investigation of concomitant medication usage between the two IBD populations indicated that neither 5-ASA nor any of the other less frequently used drugs reported as concomitant medications confounded the comparisons in this study.

### Example 1.2

### Blood Sampling and Processing

Blood (8 ml) was collected from each person into a Vacutainer cell preparation tube (CPT; Becton Dickinson, Franklin Lakes, N.J.) at the clinical site and shipped overnight to a central processing lab for PBMC isolation according to manufacturer's recommendations. All PBMCs analyzed in this study were processed within 24 hours after the blood draw. Prior to RNA purification, complete cell counts were performed on purified PBMCs using an ABX Pentra 60 C+ Hematology Analyzer (Irvine, Calif.) to record absolute counts and percentages of neutrophils, lymphocytes, monocytes, eosinophils, and basophils. Cell counts for one PBMC sample from a UC patient were not performed, and this profile was therefore excluded from the ANCOVA analyses described below. Expression data from this patient were included when developing and testing prediction models. Total RNA was purified from PBMCs using the RNeasy mini column protocol (Qiagen, Valencia, Calif.).

### Example 1.3

### Oligonucleotide Array Hybridization and Data Reduction

Total RNA (2 μg) was converted to biotinylated cRNA according to the Affymetrix protocol (Affymetrix, Santa Clara, Calif.). Labeled cRNA (10 μg) was fragmented and prepared for hybridization as previously described (Twine et al., supra). Biotinylated cRNA was hybridized to the Affymetrix HG-U133A human GENECHIP® array as described in the Affymetrix Technical Manual. Eleven biotinylated control transcripts ranging in abundance from 1:300,000 (3 ppm) to 1:1000 (1000 ppm) were spiked into each sample prior to hybridization to function as a standard curve (Hill et al. (2001) Genome Biol. 2(12):research0055.1-0055.13). GENECHIP® MAS 5.0 software was used to evaluate the specific hybridization intensity, compute a signal value for each probe set and make an absent/present call. The signal value for each probe set was then converted to a frequency value representative of the number of transcripts present in 10⁶ transcripts by reference to the standard curve (Hill et al., supra). Each transcript was evaluated and included in the study if it met the following two nonstringent criteria: called ‘present’ and at or above a frequency value of 10 (10 ppm) in at least one of the samples (healthy, UC or CD). 7,908 sequences met these filtering criteria and were used in the analysis.

### Example 1.4

### Analysis of Variance (ANOVA) and Analysis of Covariance (ANCOVA)

Analysis of covariance (ANCOVA) methods were used to adjust for differences in PBMC cell type composition when testing for differences in mean expression among disease groups. Separate ANCOVAs were run for each transcript, using log-transformed frequency as the response measure. The ANCOVA model included terms for disease group, gender, neutrophil percent, monocyte percent, and eosinophil percent. In the ANCOVA, for each cell type a slope describing the linear relationship between the percent of the cell type and the expression level for a particular gene was estimated, and a t-test was done to determine whether the slope was significantly different from 0 (where a slope of 0 indicates that there is no linear relationship between cell type percent and expression level).

The choice of cell types to include in the ANCOVA model was driven by consideration of 1) the degree of correlation between cell types, 2) the degree of difference among disease types in the distribution of each cell type, and 3) the magnitude of percents for each cell type. Covariates in an ANCOVA should not be highly correlated to each other. Lymphocyte percents were strongly inversely correlated with monocyte percents and with neutrophil percents, and for that reason were not included in the ANCOVA.

In addition to the overall tests for treatment group differences and cell type regression effects, pairwise comparisons of disease group means adjusted for differences in cell type percents were performed using two-sided t-tests, with the denominator of the t-statistics derived from the ANCOVA error term. Finally, because the relative distribution of females and males was also significantly distinct among the disease groups, gender was included in the ANCOVAs.

No adjustments of the raw p-values produced by the analyses described above were done to account for the large number of statistical tests performed. A fold-change filter (1.5 fold) combined with a conservative significance level of α=0.0001 were used to reduce the incidence of false-positive determinations.

### Example 1.5

### Gene Selection and Supervised Class Prediction using Microarray Expression Data

Gene selection and supervised class prediction were performed using GeneCluster version 2.0, which has been previously described (Golub et al. (1999) Science 286:531-37) and is available at www.broad.mit.edu/cancer/software/software.html. In these analyses only 4228 transcripts meeting a stringent data reduction filter (at least 50% present calls in Crohn's or UC samples, and at least 50% of the Crohn's or UC samples with frequencies greater than 10 ppm) were used. Samples within each group were randomly selected for membership in a data set (data set MB) consisting of a training set (75%) or a test set (25%) of profiles. Gene selection was performed using the training set of samples, and the classifier with the fewest genes that exhibited the highest overall accuracy of class assignment in the training set was identified by leave-one-out and four-fold cross-validation. The predictive classification model was then evaluated on samples in the test set, and the overall accuracy of class assignment for samples in the test set was reported.

For gene selection, all expression data in both the training set and test set were log transformed prior to analysis. In the training set of data, models containing increasing numbers of features (transcript sequences) were built using a two-sided approach (equal numbers of features in each class) with a S2N similarity metric that used median values for the class estimate. PBMC profiles from CD patients and UC patients were compared using a binary approach. Predictive gene classifiers containing between 2 and 200 genes in steps of 2 were evaluated by leave-one-out and four-fold cross-validation to identify the smallest predictive model yielding the most accurate class assignments. Prediction of class membership was performed using a weighted voting algorithm.

### Example 1.6

### Ingenuity Pathway Analysis

The Ingenuity Pathway Analysis (IPA) tool (Ingenuity, Mountain View, Calif.) was used to annotate IBD-associated genes, CD-specific genes and UC-specific genes obtained from ANCOVA analyses. Annotations on canonical pathways and functional categories were retrieved for these gene lists from the Gene-By-Gene View and/or using the Search IPKB (Ingenuity Pathways Knowledge Base) feature.

### Example 1.7

### Quantitative Real Time Polymerase Chain Reaction (Q-PCR) Confirmation of Microarray Results

Two 96-well plates containing peripheral blood mononuclear cell (PBMC) RNA samples from 59 patients with Crohn's Disease (CD) and 26 patients with ulcerative colitis (UC) were analyzed by Q-PCR. A total of 45 ng of each PBMC RNA sample was transferred into 96-well plates in a manner that preserved the original order of the sample. PBMC RNA samples from two patients with CD and one patient with UC did not contain sufficient RNA; subsequently, the samples from these patients were excluded from Q-PCR analysis. Each RNA sample was reverse-transcribed in a 100-μ1 reaction using the High Capacity cDNA Archive kit (Applied Biosystems, San Diego Calf.). The reaction was incubated at 25° C. for 10 minutes and then 37° C. for 2 hours and stored at −80° C. until amplification. Predesigned gene-specific TAQMAN® probe and primer sets (TAQMAN® gene expression assays, Applied Biosystems) corresponding to the GENBANK accession numbers for genes in the 12-gene classifier (i.e., the UniGene ID numbers found in Table 5, above) were used to amplify and quantitate the relative expression levels of classifying biomarkers. Also amplified and quantitated for each RNA sample were the expression levels of four housekeeping genes: (1) β2-microglobulin (β2M), (2) β-actin, (3) 18S ribosomal RNA (18S), and (4) glyceraldehydes-phosphate dehydrogenase (GAPDH). Applied Biosystems (ABI) IDs of TAQMAN® probes and primers used for each transcript of interest are indicated in Table 8.

TABLE 8
ABI IDs of TAQMAN probes and primers for each targeted gene.

| ABI ID | Gene Name | Symbol | Unigene ID |
|---|---|---|---|
| Hs99999901_s1 | 18S rRNA | 18S | |
| Hs00194353_m1 | lipocalin 2 (oncogene 24p3) | LCN2 | Hs.204238 |
| Hs00271778_m1 | mutL homolog 3 (E. coli) | MLH3 | Hs.279843 |
| Hs00190538_m1 | serum deprivation response | SDPR | Hs.26530 |
| | (phosphatidylserine binding protein) | | |
| Hs00269023_s1 | histone 2, H2be | HIST2H2BE | Hs.2178 |
| Hs00740275_s1 | histone 1, H3h | HIST1H3H | Hs.70937 |
| Hs00171085_m1 | chemokine (C—X—C motif) ligand 5 | CXCL5 | Hs.89714 |
| Hs00173978_m1 | integrin, beta 3 (platelet | ITGB3 | Hs.87149 |
| | glycoprotein IIIa, antigen CD61) | | |
| Hs00415042_m1 | immunoglobulin kappa constant | IGKC | Hs.406565 |
| Hs00174778_m1 | protein tyrosine phosphatase, | PTPRCAP | Hs.155979 |
| | receptor type, C-associated | | |
| | protein | | |
| Hs00157878_m1 | granzyme K (serine protease, | GZMK | Hs.3066 |
| | granzyme 3; tryptase II) | | |
| Hs00187842_m1 | beta-2-microglobulin | B2M | |
| Hs99999903_m1 | actin, beta | ACTB | |
| Hs99999905_m1 | glyceraldehyde-3-phosphate | GAPDH | |
| | dehydrogenase | | |
| Hs00413854_g1 | immunoglobulin heavy constant | IGHG1 | |
| | gamma 1 (G1m marker) | | |
| Hs00203983_m1 | mitochondrial ribosomal protein | MRPS28 | |
| | S28 | | |

Quantitative real time PCR for each transcript of interest was performed in 96-well fast block optical reaction plates in a 25-μ1 reaction volume (containing 1X TAQMAN® Fast Universal Master Mix, 1X TAQMAN® gene expression assay, and 2.25 ng of cDNA) using an ABI 7900HT sequence detection system (Applied Biosystems, San Francisco, Calif.). Negative control samples of DEPC water only (no template control; NTC) and positive control samples of human leukopack RNA were included on each 96-well plate and for each gene-specific TAQMAN® probe and primer set. Default ABI 7900HT fast block cycle conditions were as follows: 95° C. for 20 seconds, 40 cycles of 95° C. for 1 second, and 60° C. for 20 seconds. The classifying biomarkers assayed in this manner are listed in Table 9.

TABLE 9
Target genes assayed

| | |
|---|---|
| IgHg1 | Immunoglobulin heavy constant gamma 1 |
| | (also IgHg3) |
| IgKc | Immunoglobulin kappa constant |
| 28S | Human 28S ribosomal RNA 5′ region |
| PTP, C-assoc | Protein tyrosine phosphatase, receptor type, |
| | C-associated protein |
| Granzyme K | Granzyme K (Serine protease, granzyme 3; |
| | tryptase II) |
| mutL homolog 3 | mutL homolog 3 (E. coli) |
| Lipocalin-2 | Lipocalin 2 (oncogene 24p3) |
| CXCL5 | Chemokine (C—X—C motif) ligand 5 |
| serum dep response | Serum deprivation response |
| | (phosphatidylserine binding protein) |
| Histone 3K | H3 histone family, member K |
| Integrin beta-3 | Integrin, beta 3 (platelet glycoprotein IIIa, |
| | antigen CD 61) |
| Histone 2BQ | H2B histone family, member Q |

Acceptance criteria were: (1) undetectable amplification in NTC samples for each primer pair of interest, and (2) detectable gene-specific amplification in leukopack RNA positive control samples for each primer pair of interest. Cycle threshold (Ct) values for each amplification reaction were recorded for each classifying biomarker and each of the four housekeeping genes. To normalize, the differences between cycle thresholds for target genes and each of the four housekeeping genes (ΔCt) in each of the PBMC samples were calculated, and the average fold change in expression between UC and CD was calculated by the following formula: average fold difference=2^{(ΔCtUC-ΔCtCD)} or 2^{(ΔCtc-ΔCtUC)} as appropriate.

### Example 1.8

### Supervised Class Prediction using Q-PCR Expression Values

Parametric (linear), nonparametric k=3 nearest neighbor, and nonparametric k=10 nearest neighbor class assignment methods of discriminant analysis were applied to each of three data sets each divided into a training and a test subset (Data set MB (described above in Example 1.5) and two alternative random allocations of the same data to training and test subsets (Data set 1, and Data set 2)) of classifying biomarker expression levels (i.e., cycle threshold values) normalized with each of four housekeeping genes as obtained from Q-PCR (described above in Example 1.7). In all three data sets, RNA expression levels from PBMC samples from 57 patients with Crohn's Disease (CD) and 25 patients with ulcerative colitis (UC) were analyzed (43 CD plus 19 UC samples were used for training, and 14 CD plus 6 UC samples for testing). Similarly, full model, backward, forward, and stepwise methods of logistic analysis with significance levels set at p=0.05 or p=0.15 were performed on the three data sets of classifying biomarker expression levels normalized with the housekeeping gene 18S. Training set and test set accuracy, sensitivity, specificity, positive perspective value (PPV), and negative perspective value (NPV) of the classifications were calculated and compared using SASS 8.2 (Cary, NC) and SPOTFIRE® DECISIONSITE™ 8.0 (Somerville, Mass.) software. Finally, accuracy of classifiers generated using linear discriminant analysis of the ΔCts for the 12 classifying biomarkers normalized by the cycle threshold of the housekeeping gene 18S was compared to accuracy of classifiers generated using logistic analysis of the same ΔCts for twenty data sets with random allocations to training and test subsets (each containing 43 CD plus 19 UC samples for training, and 14 CD plus 6 UC samples for testing).

### Example 2

### Example 2.1

### Cellular Composition of Purified PBMC Samples from Healthy Subjects, Crohn's Disease Patients, and Ulcerative Colitis Patients

Prior to the expression-profiling portion of the study, the cellular compositions of the purified PBMC pellets from subjects in all three groups (healthy subjects, patients with CD, and patients with UC) were measured before RNA isolation. Table 10 shows the percentages of basophils, eosinophils, lymphocytes, monocytes and neutrophils in the PBMC samples.

TABLE 10
The number of samples taken from patients with IBD, in the form of
Crohn's disease (CD) or ulcerative colitis (UC), and normal disease-free
individuals (C) and average percent (%) cell types in samples

| | | | | C vs. IBD | CD vs. UC |
|---|---|---|---|---|---|
| Type | CD | UC | C | p-value¹ | p-value¹ |
| Number of | 59 | 25 | 42 | | |
| Samples | | | | | |
| Basophil % | 0.33 | 0.30 | 1.05 | 0.012 | 0.93 |
| Eosinophil % | 1.10 | 0.91 | 0.37 | 0.0003 | 0.37 |
| Lymphocyte % | 52.20 | 59.58 | 78.90 | <0.0001 | 0.056 |
| Monocyte % | 29.41 | 27.63 | 14.65 | <0.0001 | 0.52 |
| Neutrophil % | 14.96 | 10.58 | 5.00 | <0.0001 | 0.035 |

¹p-value calculated using a two-sided t-test, with t-statistic based on ANOVA error estimate.

The cellular composition of PBMC samples was significantly different (p<0.05) in the comparison of PBMCs from healthy subjects to those from IBD patients. The overall percentages of basophils and lymphocytes were significantly lower in PBMCs from patients with IBD, while the percentages of eosinophils, monocytes and neutrophils were significantly elevated in PBMCs from IBD patients. Previous studies have noted elevations in neutrophils via similar purification processes, which are due to changes in sedimentation density that appear to be related to alterations in their activation state in the peripheral blood of advanced cancer patients (Schmielau and Finn (2001) Cancer Res. 61:4756-60). The selective elevation in eosinophils, monocytes and neutrophils may be a disease-related activation event captured by the CPT-based PBMC isolation process, since cell compositions of whole blood was not significantly different between groups (data not shown).

In contrast, basophil, eosinophil, and monocyte proportions were not significantly distinct (p<0.05) between CD and UC PBMC samples. In the comparison of the two IBD groups, only neutrophils were significantly different (11% vs. 15%, p=0.035).

### Example 2.2

### Expression Level Differences in PBMCs from All IBD Patients Compared to Healthy Controls

To identify disease-associated genes that are not apparently associated with differences in cell composition, an analysis of covariance (ANCOVA) was used to identify differentially expressed transcripts while taking into account variation in cell composition among the PBMC samples. ANCOVAs were run for the 7908 transcripts that passed the standard expression level filter and the percentage of eosinophils, monocytes, and neutrophils were included as covariates.

The choice of which cell types to include was governed in part by the fact that covariates in an ANCOVA should not be highly correlated to each other. Lymphocyte percents were strongly inversely correlated with monocyte percents and with neutrophil percents, and for that reason were not included in the ANCOVA. For each cell type, a slope describing the linear relationship between the percent of the cell type and the expression level for a particular gene was estimated, and a t-test was done to determine whether the slope was significantly different from 0 (where a slope of 0 indicates that there is no linear relationship between cell type percent and expression level). Finally, because the relative distribution of females and males was also significantly distinct among the disease groups, gender was included in the ANCOVAs to identify transcripts that appeared gender-specific rather than related to disease status.

By the ANCOVA analysis, the levels of 220 transcripts were greater than 1.5 fold different between Crohn's disease and healthy PBMCs and possessed an unadjusted p-value in the pairwise comparison based on the ANCOVA of less than 0.0001, and the levels of 120 transcripts were significantly different between UC and healthy PBMCs using the same criteria as above. Forty-five of these sequences were differentially expressed in both UC and CD PBMCs, and these common PBMC- and IBD-associated transcripts changed in the same direction in both diseases compared to healthy levels (Table 1, above).

An additional filter was applied to the remaining gene sets to identify PBMC transcripts that appear differentially expressed in only one disease state. Of the 220 transcripts that were CD-associated (≧1.5 fold change, p<0.0001), a total of 67 sequences were not significantly altered in the UC PBMCs versus healthy comparison (p>0.05) and therefore appear to be CD-specific. The 67 CD-specific PBMC sequences, i.e., CD biomarkers, are presented in Table 2, above. Of the 120 transcripts that were UC-associated (≧1.5 fold change, p<0.0001), a total of 22 sequences were not significantly altered in the CD PBMCs versus the healthy comparison (p>0.05) and therefore appear to be UC-specific. The 22 UC-specific PBMC sequences, i.e., UC biomarkers, are presented in Table 3, above.

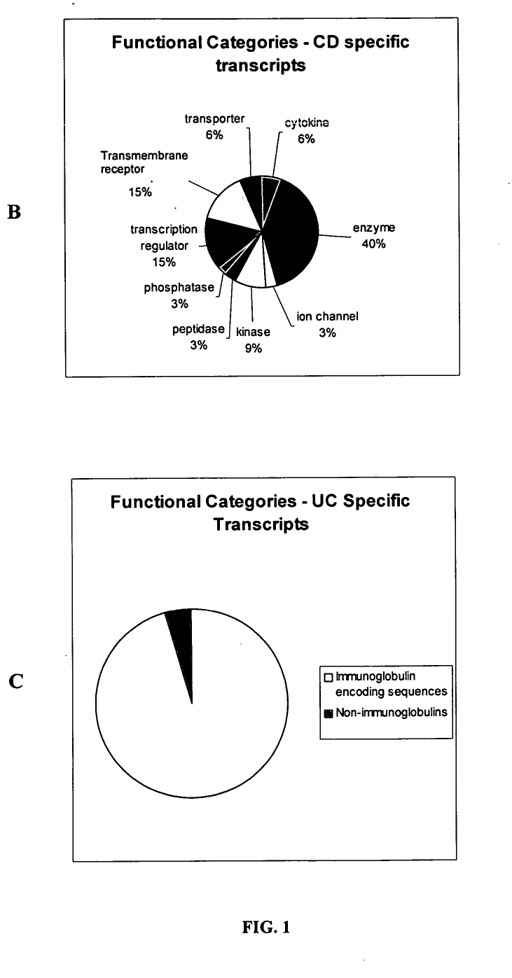

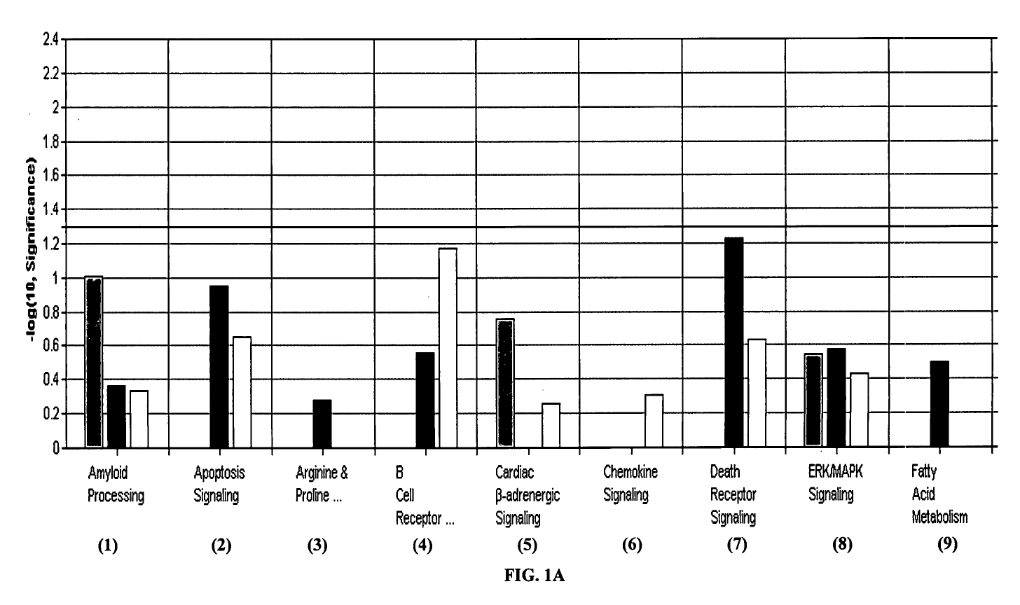

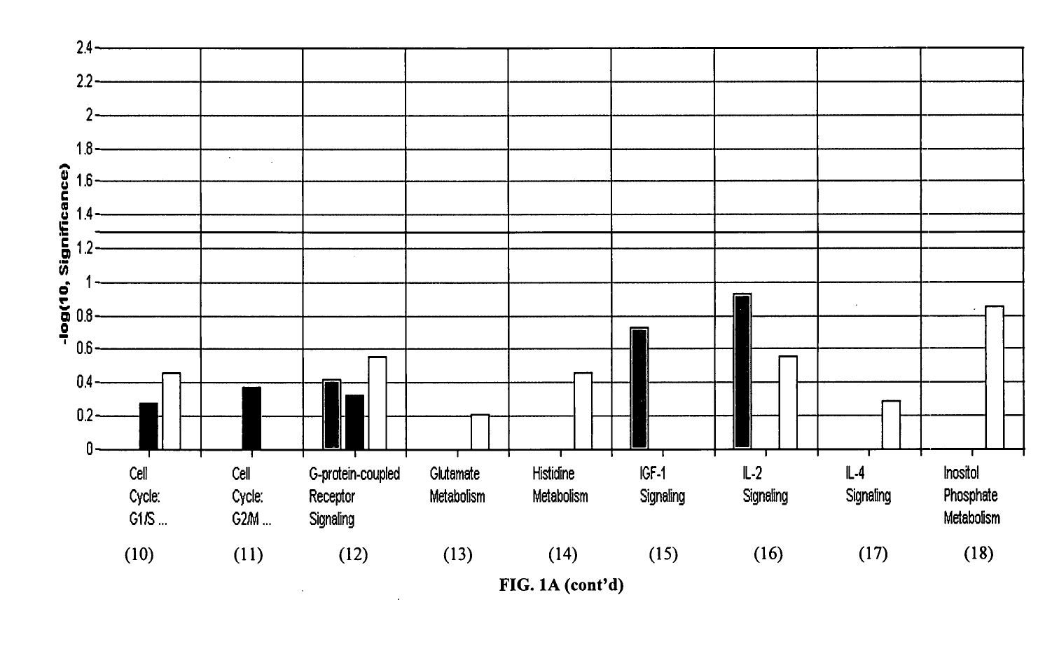

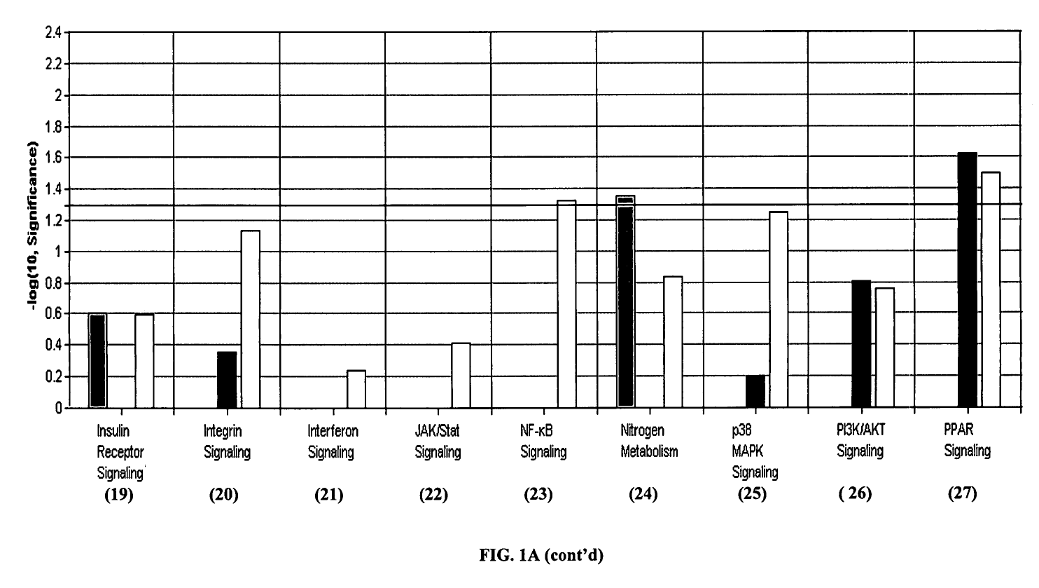

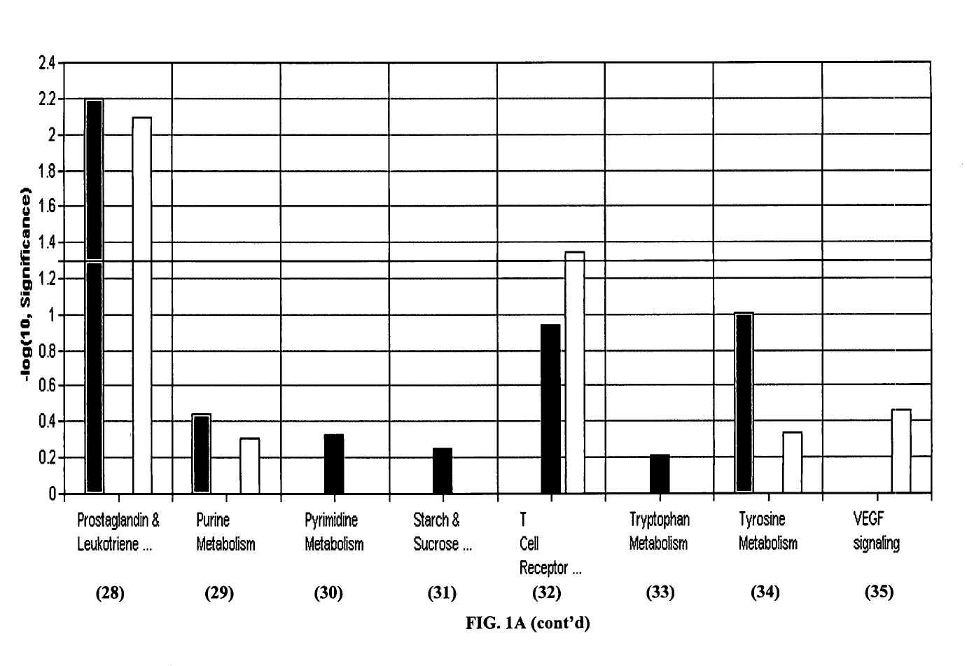

The canonical gene pathways bearing the greatest likelihood of significant overrepresentation are summarized for each comparison in FIG. 1A. In this analysis, transcripts involved in the canonical category of prostaglandin metabolism were significantly overrepresented in the CD gene signature, while transcripts encoding proteins involved in the canonical categories of apoptosis and B cell signaling appear overrepresented in the UC gene signature. FIG. 1B summarizes the diverse functional categories encompassed by the transcripts differentially expressed in Crohn's disease relative to healthy controls. Major functional categories upregulated in CD PBMCs included enzymes involved in prostaglandin metabolism, transcription regulators and transmembrane receptors, including several integrin isoforms. Finally, FIG. 1C summarizes the abundant overrepresentation of immunoglobulin constant regions that was unique to the UC PBMC gene expression signature.

### Example 2.3

### Identification of Gene Signatures Discriminating Crohn's Disease and Ulcerative Colitis

Since the main goal in the present study was to determine whether gene expression patterns in PBMCs of patients with CD and UC were sufficiently distinct to enable classification on the basis of gene expression profiles in PBMCs alone, a direct comparison of gene expression signatures between the two diseases was performed. ANCOVA comparison of CD versus UC PBMC profiles identified 49 transcripts that were present at significantly different levels between PBMCs of CD and UC patients (≧1.5 fold difference, p<0.0001). These CDvUC biomarkers are listed in Table 4, above.

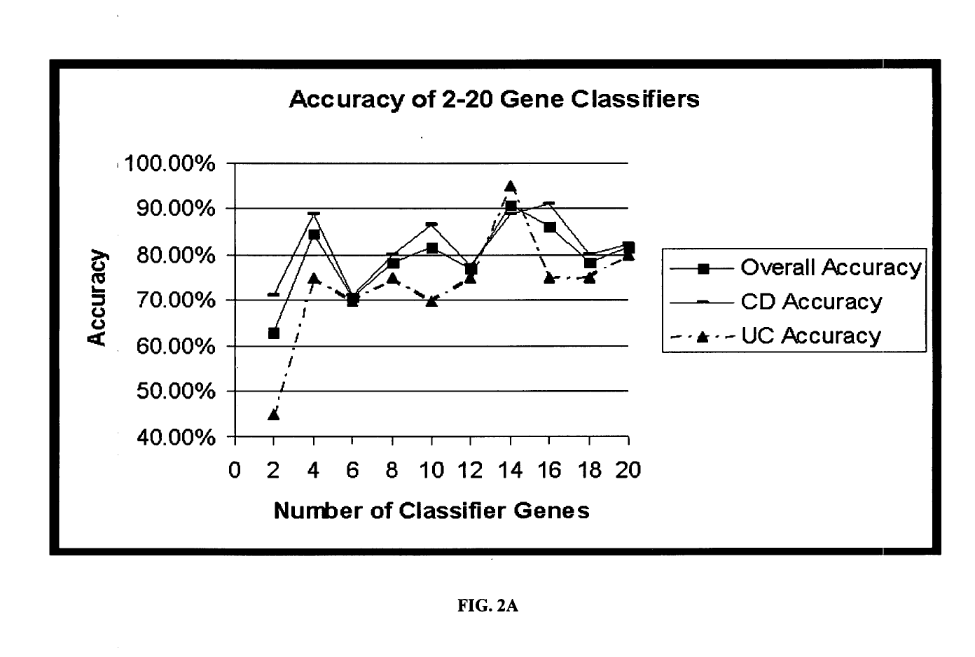

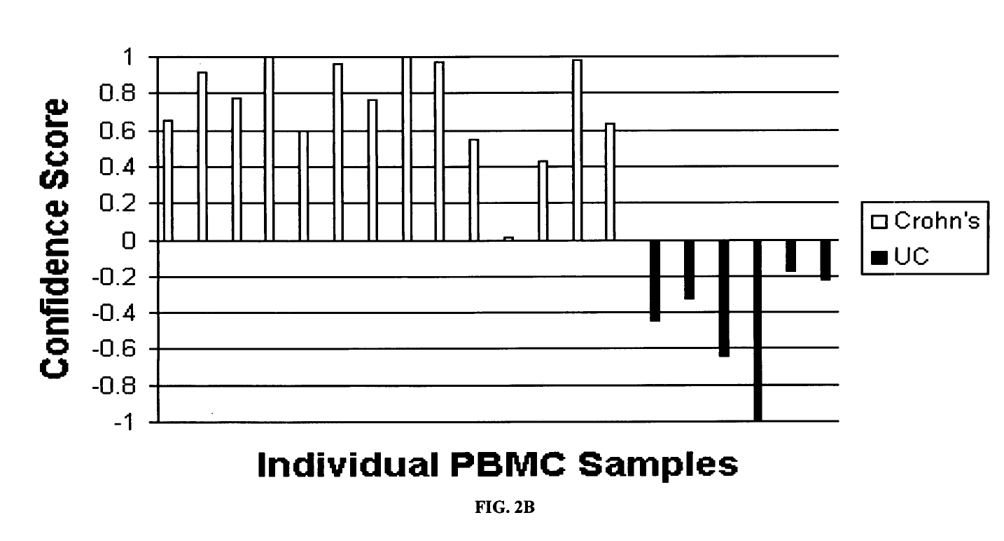

Based on the ANCOVA results indicating significant differences in direct comparison of CD and UC PBMC gene signatures, a supervised class prediction approach was employed to identify the smallest set of informative sequences capable of disease-specific classification. PBMC samples from the IBD patients were randomized into a training set composed of 44 CD and 20 UC profiles and a test set composed of 15 CD and 6 UC profiles. The relative overall accuracy, accuracy of CD classification, and accuracy of UC classification for a panel of gene classifiers of increasing size was determined (FIG. 2A). As shown in FIG. 2A, a panel consisting of two gene classifiers (i.e., lipocalin 2 and IgHg3) provided 64% accuracy as evaluated by four-fold cross validation (FIG. 2A). The smallest predictive model with the highest overall accuracy (91%) that distinguished between UC and CD PBMC profiles, as evaluated by four-fold cross-validation of the training set (FIG. 2A), was a 14-sequence (12-gene) classifier (Table 5, above). The 14-sequence classifier had a 94% overall accuracy as evaluated by leave-one-out cross validation (data not shown). The gene classifiers in Table 5 are listed in descending order of signal-to-noise ratio; i.e., of the classifying biomarkers upregulated in patients with Crohn's disease and listed in Table 5, lipocalin 2 (classifier gene no. 1) had the highest signal-to-noise ratio and integrin beta-3 (classifier gene no. 7) had the lowest signal-to-noise ratio, and of the classifying biomarkers upregulated in patients with ulcerative colitis and listed in Table 5, IgHg1 (classifier gene no. 8) had the highest signal-to-noise ratio and IgKc (classifier gene no. 14) had the lowest signal-to-noise ratio. Increasing the size of the classifier set did not increase accuracy above this level (FIG. 2A). This 12-gene classifier was used to assign class membership to the 14 CD profiles and 6 UC profiles withheld for the test set (FIG. 2B). Using this predictive model, all samples in the test set were correctly classified as clinically diagnosed. Only one individual in each group possessed a confidence score of less than 0.2 using this classifier, indicating the relatively high confidence with which these calls were made by the weighted voting algorithm. These results demonstrate the potential applicability of utilizing PBMC expression profiles to aid in the molecular diagnosis of CD and UC.

### Example 2.4

### Quantitative Real Time Reverse Transcriptase Polymerase Chain Reaction (Q-PCR) Confirmation of Microarray Observations

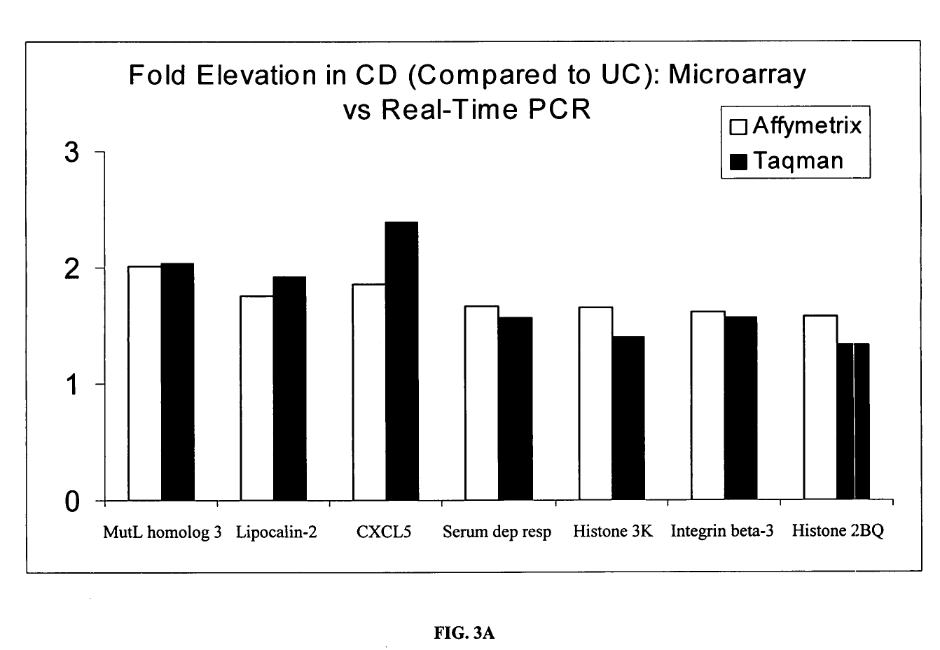

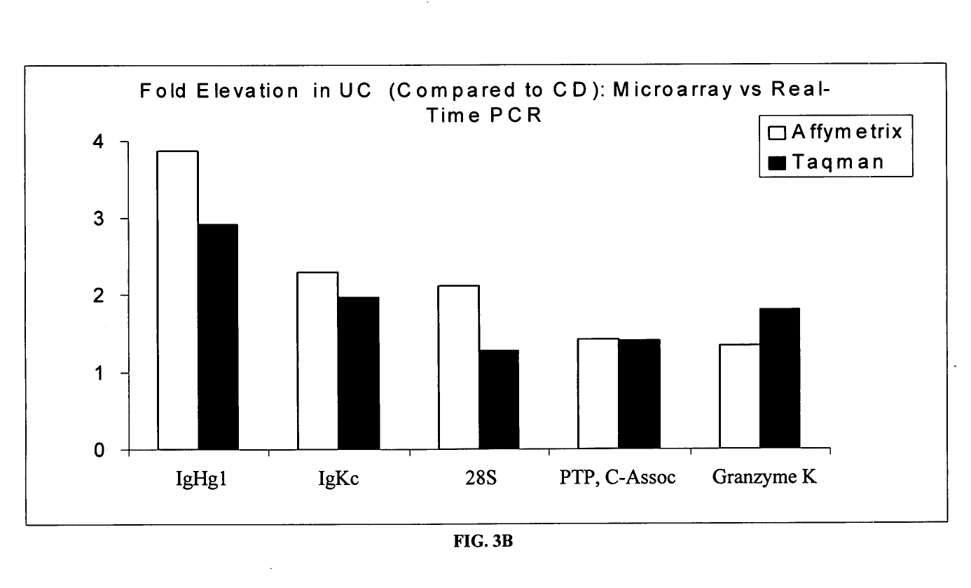

Despite the classifier set's accuracy for nearest-neighbor-based class assignment in a data set of expression levels obtained from microarray analysis, the average fold changes of transcripts in the CD/UC classifiers were relatively low. Therefore, quantitative real time PCR (Q-PCR) was performed to confirm the relative expression observed by Affymetrix microarray technology for CD and UC samples in this study. Four separate housekeeping genes for normalization of the target genes were used: β2-microglobulin (β2M), β-actin, GAPDH, and 18S ribosomal RNA (18S). All CD and UC RNA samples in the study were converted to cDNA using the same reverse-transcription cocktail and procedure. Comparison of average fold changes calculated by microarray and real-time PCR using β2-microglobulin are presented in FIG. 3, and relative fold changes for all 12 classifying genes using each of the four housekeeping genes as normalizers were extremely concordant (Table 11).

TABLE 11
Relative fold changes after normalization.
Elevated in UC (Compared to CD):

| | | | | | PTP, | |
|---|---|---|---|---|---|---|
| Test | Normalization | IgHg1 | IgKc | 28S | C-assoc | Granzyme K |
| Affy | Scaled | 3.87 | 2.30 | 2.11 | 1.43 | 1.35 |
| | Frequency | | | | | |
| Q-PCR | β2M | 3.11 | 2.04 | 1.32 | 1.47 | 1.85 |
| | β-actin | 3.05 | 2.00 | 1.30 | 1.44 | 1.81 |
| | GAPDH | 2.83 | 1.86 | 1.21 | 1.34 | 1.69 |
| | 18S | 2.68 | 1.98 | 1.31 | 1.40 | 1.86 |

Elevated in CD (Compared to UC):

| | | | | | serum | | | |
|---|---|---|---|---|---|---|---|---|
| | | | | | dep | | Integrin | Histone |
| Test | Normalization | mutL 3 | Lipocalin-2 | CXCL5 | response | Histone 3K | beta-3 | 2BQ |
| Affy | Scaled | 2.01 | 1.75 | 1.85 | 1.66 | 1.65 | 1.62 | 1.57 |
| | Frequency | | | | | | | |
| Q-PCR | β2M | 1.93 | 1.84 | 2.28 | 1.49 | 1.32 | 1.49 | 1.27 |
| | β-actin | 1.97 | 1.88 | 2.33 | 1.52 | 1.35 | 1.52 | 1.29 |
| | GAPDH | 2.12 | 2.02 | 2.50 | 1.64 | 1.45 | 1.64 | 1.39 |
| | 18S | 2.13 | 1.96 | 2.44 | 1.59 | 1.47 | 1.61 | 1.36 |

On the basis of these results, of the 12 transcripts originally identified as CD/UC discriminator genes (i.e., classifying biomarkers), only the 28S rRNA fragment appears to have been significantly overestimated by microarray hybridization.

### Example 2.5

### Accurate Class Prediction using Expression Values Obtained by Q-PCR

Linear discriminant analysis (LDA) of ΔCts for the twelve classifying biomarkers (listed in Tables 5 and 8) normalized with each of four housekeeping genes (β2M, β-actin, GAPDH, and 18S) was compared to k-NN discriminant analysis of the same ΔCts for three data sets (data set MB, data set 1, data set 2) consisting of 43 PBMC RNA samples isolated from patients with CD and 19 PBMC RNA samples isolated from patients with UC in the training set, and 14 PBMC RNA samples isolated from patients with CD and 6 PBMC RNA samples isolated from patients with UC in the test set.

The accuracy, sensitivity, specificity, PPV, and NPV measures of classification performance for parametric (linear), nonparametric k=3 nearest neighbor, or nonparametric k=10 nearest neighbor methods of discriminant analysis for the three data sets (data set MB, data set 1, data set 2) of expression levels of classifying biomarkers normalized to each of the four housekeeping (β2-microglobulin (β2M), β-actin, GAPDH, and 18S ribosomal RNA (18S)) genes are shown in Table 13.

TABLE 13

| Housekeeping | | | | | | | |
|---|---|---|---|---|---|---|---|
| Gene | Data set | Method | Accuracy | Sensitivity | Specificity | PPV | NPV |
| β2M | set MB | parametric | 0.833 | 1.000 | 0.667 | 0.875 | 1.000 |
| β2M | set MB | K-NN k = 3 | 0.762 | 0.857 | 0.667 | 0.857 | 0.667 |
| β2M | set MB | K-NN k = 10 | 0.750 | 1.000 | 0.500 | 0.824 | 1.000 |
| β-actin | set MB | parametric | 0.833 | 1.000 | 0.667 | 0.875 | 1.000 |
| β-actin | set MB | K-NN k = 3 | 0.798 | 0.929 | 0.667 | 0.867 | 0.800 |
| β-actin | set MB | K-NN k = 10 | 0.750 | 1.000 | 0.500 | 0.824 | 1.000 |
| GAPDH | set MB | parametric | 0.750 | 1.000 | 0.500 | 0.824 | 1.000 |
| GAPDH | set MB | K-NN k = 3 | 0.762 | 0.857 | 0.667 | 0.857 | 0.667 |
| GAPDH | set MB | K-NN k = 10 | 0.750 | 1.000 | 0.500 | 0.824 | 1.000 |
| 18S | set MB | parametric | 0.917 | 1.000 | 0.833 | 0.933 | 1.000 |
| 18S | set MB | K-NN k = 3 | 0.845 | 0.857 | 0.833 | 0.923 | 0.714 |
| 18S | set MB | K-NN k = 10 | 0.917 | 1.000 | 0.833 | 0.933 | 1.000 |
| β2M | set 1 | parametric | 0.964 | 0.929 | 1.000 | 1.000 | 0.857 |
| β2M | set 1 | K-NN k = 3 | 0.821 | 0.643 | 1.000 | 1.000 | 0.545 |
| β2M | set 1 | K-NN k = 10 | 0.798 | 0.929 | 0.667 | 0.867 | 0.800 |
| β-actin | set 1 | parametric | 0.964 | 0.929 | 1.000 | 1.000 | 0.857 |
| β-actin | set 1 | K-NN k = 3 | 0.821 | 0.643 | 1.000 | 1.000 | 0.545 |
| β-actin | set 1 | K-NN k = 10 | 0.845 | 0.857 | 0.833 | 0.923 | 0.714 |
| GAPDH | set 1 | parametric | 0.929 | 0.857 | 1.000 | 1.000 | 0.750 |
| GAPDH | set 1 | K-NN k = 3 | 0.786 | 0.571 | 1.000 | 1.000 | 0.500 |
| GAPDH | set 1 | K-NN k = 10 | 0.845 | 0.857 | 0.833 | 0.923 | 0.714 |
| 18S | set 1 | parametric | 0.845 | 0.857 | 0.833 | 0.923 | 0.714 |
| 18S | set 1 | K-NN k = 3 | 0.810 | 0.786 | 0.833 | 0.917 | 0.625 |
| 18S | set 1 | K-NN k = 10 | 0.845 | 0.857 | 0.833 | 0.923 | 0.714 |
| β2M | set 2 | parametric | 0.774 | 0.714 | 0.833 | 0.909 | 0.556 |
| β2M | set 2 | K-NN k = 3 | 0.738 | 0.643 | 0.667 | 0.818 | 0.444 |
| β2M | set 2 | K-NN k = 10 | 0.881 | 0.929 | 0.833 | 0.929 | 0.833 |
| β-actin | set 2 | parametric | 0.774 | 0.714 | 0.833 | 0.909 | 0.556 |
| β-actin | set 2 | K-NN k = 3 | 0.702 | 0.571 | 0.833 | 0.889 | 0.455 |
| β-actin | set 2 | K-NN k = 10 | 0.762 | 0.857 | 0.667 | 0.857 | 0.667 |
| GAPDH | set 2 | parametric | 0.810 | 0.786 | 0.833 | 0.917 | 0.625 |
| GAPDH | set 2 | K-NN k = 3 | 0.667 | 0.500 | 0.833 | 0.875 | 0.417 |
| GAPDH | set 2 | K-NN k = 10 | 0.810 | 0.786 | 0.833 | 0.917 | 0.625 |
| 18S | set 2 | parametric | 0.845 | 0.857 | 0.833 | 0.923 | 0.714 |
| 18S | set 2 | K-NN k = 3 | 0.810 | 0.786 | 0.833 | 0.917 | 0.625 |
| 18S | set 2 | K-NN k = 10 | 0.845 | 0.857 | 0.833 | 0.923 | 0.714 |

The discriminant classification worked best on data set MB regardless of the method used, suggesting that performance of each method is related to each data set analyzed (Table 13). Both parametric and nonparametric with k=10 nearest neighbor methods worked similarly, although on average both worked better than nonparametric with k=3 nearest neighbor method (Table 13).

Classifying biomarkers normalized with 18S showed a systematic difference in performance among the three data sets. 18S outperformed the other housekeeping genes to some degree; analysis of classifying biomarkers normalized with 18S consistently had higher values and smaller variabilities in accuracy, sensitivity, and specificity (Table 13).

Since 18S appeared to outperform the other housekeeping genes tested, logistic analysis of the expression levels of classifying biomarkers normalized with only 18S was then performed. The choice among full model, backward, forward, or stepwise selection methods of logistic analysis had little impact on classification (data not shown). The full model method worked as well or better than the other reduced models on data set MB and data set 1 (data not shown). In the case of data set 2, models with two or three classifying biomarkers normalized with 18S (both including MLH3 and IgKC) selected by forward or stepwise methods with the significance level set to p=0.05 had better class prediction (data not shown). This finding may have been due to the fact that most of the biomarkers with atypical expression levels were included in the test set for data set 2, while the model from the forward or stepwise selection did not contain those abnormally expressed biomarkers. This variance in inclusion of biomarkers may also explain why the full model was not the best performing logistic method and why a poorer classification resulted from discriminant analysis with a model including all 12 qualifiers. The accuracy, sensitivity, and specificity of different selection methods for logistic analysis indicate that different logistic selection methods performed similarly (data not shown). For example, if one method showed better classification on the training set than the test set of a data set, or vice versa, then the results with all three other methods showed the same property.

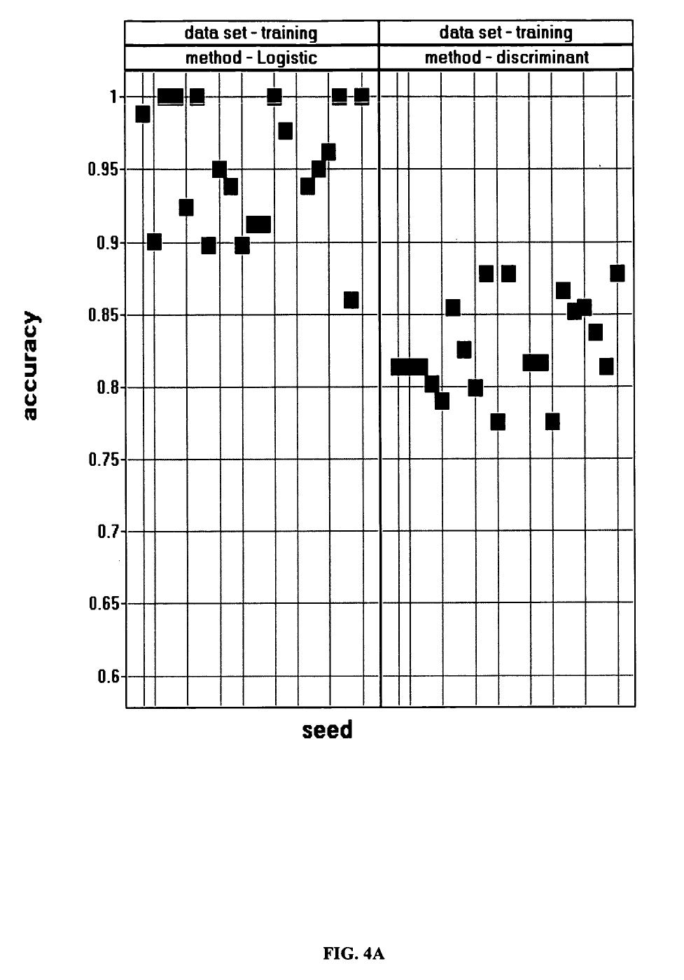

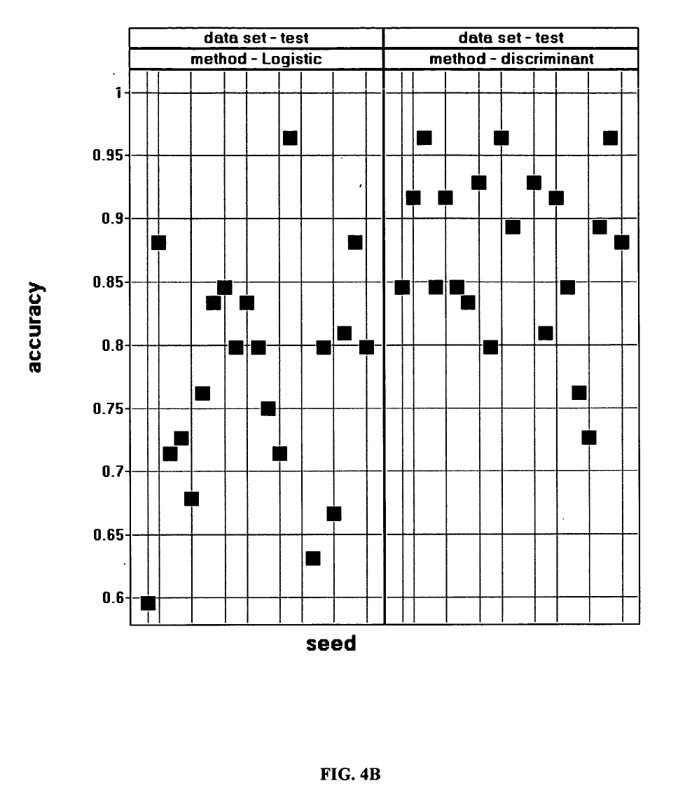

Classification by logistic analysis using a full model was then compared to classification by linear discriminant analysis using the parametric method. While data set variations between the two methods were observed, both analyses adequately performed class prediction. The class prediction using data set MB, data set 1 and data set 2 resulted in accuracy between 0.833 and 0.917, sensitivity between 0.786 and 1.000, and specificity between 0.667 and 1.000 (Table 14). However, a difference between the two methods appeared when 20 data sets were compared (FIG. 4). The logistic analysis with the full model worked better on cross-validation, whereas the discriminant analysis did better on test set classification.

TABLE 14

| | analysis | | | | | |
|---|---|---|---|---|---|---|
| data set | method | sensitivity | specificity | PPV | NPV | accuracy |
| MB | Discriminant | 0.786 | 1.000 | 1.000 | 0.667 | 0.893 |
| MB | Logistic | 1.000 | 0.667 | 0.875 | 1.000 | 0.833 |
| set 1 | Discriminant | 0.857 | 0.833 | 0.923 | 0.714 | 0.845 |
| set 1 | Logistic | 1.000 | 0.833 | 0.933 | 1.000 | 0.917 |
| set 2 | Discriminant | 0.786 | 1.000 | 1.000 | 0.667 | 0.893 |
| set 2 | Logistic | 0.857 | 0.833 | 0.923 | 0.714 | 0.845 |

### Example 3

### Discussion

The focus of the present study was an attempt to determine (1) both the commonalities and specificities of gene expression patterns in PBMCs associated with CD and UC and (2) whether disease-specific expression signatures could contribute to a molecular diagnosis of disease. Several dozen genes appear to be differentially expressed in profiles for both CD and UC patients compared with profiles for healthy subjects. Many of these genes encode nuclear proteins such as transcription regulators, and most are downregulated. Examples include NFKB2, RNA-binding factors CUGBP1 and CUGBP2, COPEB, and ELK3. Disregulated inflammatory processes common to UC and CD may be a consequence of modulation of the activity of these transcriptional regulators.

The most highly expressed gene commonly elevated in both inflammatory bowel diseases was the protease inhibitor SERPINB2 (also called PAI, plasminogen activator inhibitor, type II). Increased plasminogen activator levels have been reported in mucosal lesions of IBD patients (de Bruin et al. (1988) Thromb. Haemost. 60:262-66) and increased PAI-I was found in IBD patient plasma. Although distinct from PAI-1, PAI-2 shares enzyme specificity to both u-PA and to a lesser degree, t-TA, and elevated PAI-2 levels are reported in rheumatoid arthritis synovial fluid (Kruithof et al. (1995) Blood 86:4007-24). These findings suggest changes in components of the fibrinolytic and coagulation system(s) may contribute to an increased risk for thromboembolic complications and possibly to colitis and bleeding seen in IBD patients (de Jong et al. (1989) Gut 30:188-94). A role for PAI-2 in IBD has not been reported, but this study suggests that elevated PAI-2 RNA levels in PBMCs are associated with disease.

Multiple functional classes of transcripts appear specifically upregulated in PBMCs of CD patients, including prostaglandin metabolizing enzymes, chemokines, and transcriptional regulators. The CD-specific PBMC gene profile exhibited a proinflammatory gene expression profile that was not apparent in the UC-specific PBMC gene profile. Genes involved in prostaglandin and leukotriene metabolism, e.g., arachidonate 12-lipoxygenase (ALOX12) and prostaglandin endoperoxide synthase 1 (PTGS 1, cyclooxygenase 1), were significantly increased in PBMCs from CD patients, while prostaglandin D2 synthase (PTGDS) was decreased. These effects on the prostaglandin synthetic pathway would be expected to result in increased conversion of arachidonic acid into select prostaglandins. Although prostaglandin content is elevated in lesions of IBD patients (Schmidt et al. (1996) Hepatogastroenterology 43:1508-12), very recent evidence suggests that levels of at least one prostaglandin (PGE₂) are actually decreased in mononuclear cells of patients with CD (Trebble et al. (2004) Clin. Nutr. 23:647-55). It is unclear whether the relative elevations in transcripts encoding arachidonic acid metabolizing enzymes in PBMCs of CD patients are functionally linked to this observation, but PGE₂ has been documented as an important modulator of cytokine release from T lymphocytes derived from the gastrointestinal tract (Barrera et al. (1996) J. Cell. Physiol. 166:130-37). The upregulation of PG metabolic pathways in circulating PBMCs of Crohn's disease patients may represent alterations in cells entering/exiting the lamina propria of the intestine in this disease.

Several chemokines (C-X-C ligands 4 and 7, platelet factor 4 variant 1) were upregulated in CD. Overall there was surprisingly little overlap between transcripts identified as upregulated in the present set of CD PBMCs and those reported as upregulated in the seven CD patients analyzed by Mannick and colleagues (Mannick et al., supra). It is unknown whether this is attributable to the larger number of patients explored herein, the larger number of genes interrogated, differences in gene nomenclature, or some other confounding factor between these studies. However, the most strongly upregulated transcript in CD reported by Mannick and coworkers encoded a transforming growth factor (TGF)-β-inducible transcript (Mannick et al., supra). Here, TSC-22, a distinct TGF-β-inducible transcript, was also identified as upregulated in CD PBMCs. These observations show that upregulation of TGF-β signal transduction appears to be evident in CD PBMCs. Constitutive elevation in this pathway could result in downregulation of Smad-dependent pathways, which subsequently may result in the inhibited ability of TGF-β to terminate immune responses and in turn play a causal role in the pathogenesis of CD (Mannick et al., supra).

It is possible that a portion of the Crohn's-associated disease signature gene profile may be platelet-derived. Recent evidence has demonstrated that platelets can participate in chronic intestinal inflammation (Danese et al. (2004) Am. J. Gastroenterol. 99:938-45) and platelets copurified to a greater extent with the PBMCs isolated from CD patients in this study (data not shown). Thus, the detection of platelet factor 4 and platelet factor 4 variant 1 in the CD-associated gene signature could be attributable to elevated levels of copurified platelets in isolated PBMCs. However, other transcripts among the top 10 nonmitochondrial transcripts reported in platelets (Gnatenko et al. (2003) Blood 101:2285-93) do not appear in the present CD-associated list of transcripts, suggesting that the levels of these anucleate cells are not the sole source of these transcripts. All of the transcripts in the CD disease signature that have been previously associated with platelets are also expressed at significant levels in purified T cells, B cells, and/or monocytes (data not shown), which suggests that transcripts previously associated with platelets can originate from the mononuclear cells that were isolated and profiled in this study.

The UC-specific gene set was dominated by overexpression of immunoglobulin encoding sequences, reminiscent of the active IgG plasma cell component observed in UC patients (Farrell et al. (2002) Lancet 359:331-40). This finding is consistent with studies on B-cell receptor gene usage that have demonstrated that infiltrating lymphocytes in UC mucosa are of peripheral, rather than mucosal origin (Dunn-Walters et al. (1999) Gut 44:382-86; Thoree et al. (2002) Gut 51:44-50). IgG1 and IgG4 antibodies predominate in UC, whereas IgG2 antibodies are increased in CD (Kett and Brandtzaeg (1987) Gut 28:1013-21). The prevalence of the IgG1 type has recently been explored and shown to be specific to UC and to lead to greater opsonization of mucosal bacteria and a feed-forward maintenance of the polymorphonuclear leukocyte respiratory burst in UC (Furrie et al. (2004) Gut 53:91-98). One of the transcripts most significantly elevated in UC PBMCs in this study was annotated as immunoglobulin heavy constant gamma 3 (IgHG3). The region encompassed by this IgHG3 qualifier on the Affymetrix chip actually maps (i.e., shares 100% nucleotide identity by BLAST) to several sequences ascribed to immunoglobulin heavy constant gamma 1 (G1m marker) and has been identified as a marker of inflamed UC gastrointestinal epithelium (Warner and Dieckgraefe, supra; Lawrance et al., supra). Analysis of the expression level of IgHG3 transcripts in the peripheral blood profiles of individual patients showed that their levels may serve as a distinctive biomarker between UC and CD (data not shown). However, these results are also consistent with the previous observation that IgGI levels in serum are significantly increased in UC patients relative to serum levels of IgG1 in CD patients (Gouni-Berthold et al. (1999) Hepatogastroenterology 46:1720-23).

A significant subset of patients with inflammatory bowel disease cannot be classified by current procedures and constitute cases of “indeterminate IBD” (Winther et al. (1998) Drugs Today (Barc). 34:935-42; Bentley et al. (2002) J. Clin. PathoL. 55:955-60; Guindi and Riddell (2004) J. Clin. Pathol. 57:1233-44). Therefore, one of the main goals of the present study was to determine whether PBMC profiles in patients with UC and CD were sufficiently distinct to enable classification of these diseases. Results of class prediction analysis indicate that a gene signature in PBMCs can accurately discriminate UC and CD samples. Transcriptional differences are not due to cellular composition, since cellular compositions of PBMCs from patients appear quite similar.

Although prospective validation in a larger population would likely be performed, the disease-specific patterns identified by the invention may provide the basis of a molecular diagnosis of UC and CD, and may contribute to the diagnosis of patients classified as suffering from indeterminate IBD. It is quite possible that the proposed Th1 and Th2 natures of CD and UC, respectively, are mainly responsible for the differences in this study, and that other Th1- and Th2-based inflammatory diseases may bear similar signatures to those identified for CD and UC. Nonetheless, the PBMC profile identified herein appears to have clinical utility in IBD, because the gene classifier enables discrimination between these closely related disorders that are often difficult to distinguish and sometimes indistinguishable.

This study indicates that transcriptional profiles in circulating monocytes, T cells, and B cells may serve as a sensitive monitor of an organism's physiological state in the context of IBD. As these cells traverse various tissues, one component of the cellular reaction to the microenvironment is a transcriptional response; such a response can be quantitated through profiling. Expression patterns may reflect disease mechanism(s) of primary or secondary responses to disease pathophysiology. PBMCs, due to their transit through the body, may serve as an accessible surrogate monitor of tissues and systems that are not easily surveyed, such as though tissues and systems affected by IBD.

What is claimed is:

1. A method of diagnosing inflammatory bowel disease in a patient, the method comprising the steps of:
a. isolating a sample from the patient; and
b. detecting in the sample the normal or aberrant expression of at least one PBMC- and IBD-associated biomarker,
wherein the aberrant expression of at least one PBMC- and IBD-associated biomarker indicates that the patient may be afflicted with inflammatory bowel disease.

2. The method of claim 1, wherein the sample is a collection of peripheral blood mononuclear cells.

3. The method of claim 1, wherein the detecting step is performed with a hybridization-based assay.

4. The method of claim 1, wherein the detecting step is performed with an immunological assay.

5. The method of claim 1, wherein the detecting step is performed with a polymerase chain reaction.

6. The method of claim 5, wherein the polymerase chain reaction is a quantitative polymerase chain reaction.

7. The method of claim 1, wherein the detecting step detects the expression of a panel of PBMC- and IBD-associated biomarkers.

8. The method of claim 7, wherein the panel of PBMC- and IBD-associated biomarkers comprises at least one common biomarker.

9. The method of claim 8, wherein the panel of PBMC- and IBD-associated biomarkers comprises at least one Group I biomarker.

10. The panel of claim 9, wherein the panel of PBMC- and IBD-associated biomarkers includes PAI-2.

11. The method of claim 7, wherein the panel of PBMC- and IBD-associated biomarkers comprises at least one CD biomarker.

12. The method of claim 11, wherein the panel of PBMC- and IBD-associated biomarkers comprises at least one Group II biomarker.

13. The method of claim 11, wherein the panel of PBMC- and IBD-associated biomarkers includes ALOX12.

14. The method of claim 11, wherein the panel of PBMC- and IBD-associated biomarkers includes PTGDS.

15. The method of claim 11, wherein the panel of PBMC- and IBD-associated biomarkers includes lipocalin 2.

16. The method of claim 7, wherein the panel of PBMC- and IBD-associated biomarkers comprises at least one UC biomarker.

17. The method of claim 16, wherein the panel of PBMC- and IBD-associated biomarkers comprises at least one Group III biomarker.

18. The method of claim 17, wherein the panel of PBMC- and IBD-associated biomarkers includes IgHG3.

19. The method of claim 7, wherein the panel of PBMC- and IBD-associated biomarkers comprises at least one CDvUC biomarker.

20. The method of claim 19, wherein the panel of PBMC- and IBD-associated biomarkers comprises at least one Group IV biomarker.

21. The method of claim 7, wherein the panel of PBMC- and IBD-associated biomarkers comprises at least one classifying biomarker.

22. The method of claim 21, wherein the panel of PBMC- and IBD-associated biomarkers comprises at least one Group V biomarker.

23. The method of claim 7, wherein the panel of PBMC- and IBD-associated biomarkers comprises a set of biomarkers selected from the group consisting of the set of Group I biomarkers, the set of Group II biomarkers, the set of Group III biomarkers, the set of Group IV biomarkers, and the set of Group V biomarkers.

24. A method of diagnosing ulcerative colitis in a patient, the method comprising the steps of:
a. isolating a sample from the patient; and
b. detecting in the sample the normal or aberrant expression of at least one ulcerative colitis-associated biomarker,
wherein the aberrant expression of at least one ulcerative colitis-associated biomarker indicates that the patient may be afflicted with ulcerative colitis.

25. The method of claim 24, wherein the sample is a collection of peripheral blood mononuclear cells.

26. The method of claim 24, wherein the at least one ulcerative colitis-associated biomarker is selected from the group consisting of the PBMC- and IBD-associated biomarkers categorized as Group III biomarkers.

27. The method of claim 26, wherein the at least one ulcerative colitis-associated biomarker includes IgHG3.

28. The method of claim 24, wherein the detecting step is performed with a hybridization-based assay.

29. The method of claim 24, wherein the detecting step is performed with an immunological assay.

30. The method of claim 24, wherein the detecting step is performed with a polymerase chain reaction.

31. The method of claim 30, wherein the polymerase chain reaction is a quantitative polymerase chain reaction.

32. The method of claim 24, wherein the detecting step detects the expression of a panel of PBMC- and IBD-associated biomarkers.

33. A method of diagnosing Crohn's disease in a patient, the method comprising the steps of:
a. isolating a sample from the patient; and
b. detecting in the sample the normal or aberrant expression of at least one Crohn's disease-associated biomarker,
wherein the aberrant expression of at least one Crohn's disease-associated biomarker indicates that the patient may be afflicted with Crohn's disease.

34. The method of claim 33, wherein the sample is a collection of peripheral blood mononuclear cells.

35. The method of claim 33, wherein the at least one Crohn's disease-associated biomarker is selected from the group consisting of the PBMC- and IBD-associated biomarkers categorized as Group II biomarkers.

36. The method of claim 33, wherein the detecting step is performed with a hybridization-based assay.

37. The method of claim 33, wherein the detecting step is performed with an immunological assay.

38. The method of claim 33, wherein the detecting step is performed with a polymerase chain reaction.

39. The method of claim 38, wherein the polymerase chain reaction is a quantitative polymerase chain reaction.

40. The method of claim 33, wherein the detecting step detects the expression of a panel of PBMC- and IBD-associated biomarkers.

41. A method of distinguishing between a diagnosis of ulcerative colitis and a diagnosis of Crohn's disease in a patient, the method comprising the steps of:
a. isolating a sample from the patient; and
b. detecting in the sample the normal or aberrant expression of at least one classifying biomarker,
wherein the aberrant expression of at least one classifying biomarker associated with distinguishing patients with Crohn's disease indicates that the patient may be afflicted with Crohn's disease, or
wherein the aberrant expression of at least one classifying biomarker associated with distinguishing patients with ulcerative colitis indicates that the patient may be afflicted with ulcerative colitis.

42. The method of claim 41, wherein the sample is a collection of peripheral blood mononuclear cells.

43. The method of claim 41, wherein the at least one classifying biomarker is selected from the group consisting of the classifying biomarkers categorized as Group V biomarkers.

44. The method of claim 41, wherein the detecting step is performed with a hybridization-based assay.

45. The method of claim 41, wherein the detecting step is performed with an immunological assay.

46. The method of claim 41, wherein the detecting step is performed with a polymerase chain reaction.

47. The method of claim 46, wherein the polymerase chain reaction is a quantitative polymerase chain reaction.

48. The method of claim 41, wherein the detecting step comprises detecting in the sample the normal or aberrant expression of a panel of classifying biomarkers, and wherein the panel of classifying biomarkers comprises the immunoglobulin heavy constant gamma 1, immunoglobulin kappa constant, human 28S ribosomal RNA 5′protein tyrosine phosphatase receptor type C-associated protein, granzyme K, mutL homolog 3, lipocalin 2, CXCL5, serum deprivation response phosphatidylserine binding protein, H3 histone family member K, integrin beta 3 (platelet glycoprotein IIIa, antigen CD 61), and H2B histone family member Q biomarkers.

49. The method of claim 41, wherein the detecting step comprises detecting in the sample the normal or aberrant expression of a panel of classifying biomarkers, and wherein the panel of classifying biomarkers comprises at least 2 classifying biomarkers selected from the group consisting of the immunoglobulin heavy constant gamma 1, immunoglobulin kappa constant, human 28S ribosomal RNA 5′region, protein tyrosine phosphatase receptor type C-associated protein, granzyme K, mutL homolog 3, lipocalin 2, CXCL5, serum deprivation response phosphatidylserine binding protein, H3 histone family member K, integrin beta 3 (platelet glycoprotein IIIa, antigen CD 61), and H2B histone family member Q biomarkers.

50. The method claim 41, wherein the detecting step comprises detecting in the sample the normal or aberrant expression of a panel of classifying biomarkers, and wherein the panel of classifying biomarkers comprises at least eight classifying biomarkers selected from the group consisting of the immunoglobulin heavy constant gamma 1, immunoglobulin kappa constant, human 28S ribosomal RNA 5′region, protein tyrosine phosphatase receptor type C-associated protein, granzyme K, mutL homolog 3, lipocalin 2, CXCL5, serum deprivation response phosphatidylserine binding protein, H3 histone family member K, integrin beta 3 (platelet glycoprotein IIIa, antigen CD 61), and H2B histone family member Q biomarkers.
